SEQUENCE LISTING (1 sequence)

SEQ ID NO: 1 - protein, 19 aa, HOMO SAPIENS
DLDLEMLAPYIPMDDDFQL

(19) United States
(12) Patent Application Publication
(10) Pub. No.: US 2017/0247336 A1
(43) Pub. Date: Aug. 31, 2017

(54) SUBSTITUTED PYRIMIDINES AS INHIBITORS OF HIF PROLYL HYDROXYLASE

(71) Applicant: MERCK SHARP & DOHME CORP., RAHWAY, NJ (US)

(72) Inventors: FEZ UJJAINWALLA, HOBOKEN, NJ (US); JOHN QIANG TAN, PRINCETON, NJ (US); QUN DANG, WESTFIELD, NJ (US); CHRISTOPHER J. SINZ, MIDDLETOWN, NJ (US); ALEJANDRO CRESPO, EDISON, NJ (US); MING WANG, BELLE MEAD, NJ (US); YILI CHEN, HILLSBOROUGH, NJ (US); JIAQIANG CAI, SHANGHAI (CN); FAN WU, SHANGHAI (CN); XIAOXING DU, SHANGHAI (CN)

(73) Assignee: MERCK SHARP & DOHME CORP., RAHWAY, NJ (US)

(21) Appl. No.: 15/517,557

(22) PCT Filed: Oct. 8, 2015
(86) PCT No.: PCT/US2015/054643

§ 371 Date: Apr. 7, 2017

(30) Foreign Application Priority Data
Oct. 10, 2014 (CN) PCT/CN2014/088318

(51) Int. Cl. C07D 239/36; C07D 401/12; C07D 405/12; C07D 403/12

(52) U.S. Cl. CPC C07D 239/36; C07D 403/12; C07D 401/12; C07D 405/12

(57) ABSTRACT

The present invention concerns compounds of Formula I that inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### BACKGROUND OF THE INVENTION

The insufficient delivery of oxygen to cells and tissues is associated with anemia, which is defined as a deficiency in the blood's oxygen-carrying capacity, and ischemia, in which restrictions in blood supply are caused by a constriction or blockage of blood vessels. Anemia can be caused by the loss of red blood cells (hemorrhage), excessive red blood cell destruction (hemolysis) or deficiencies in erythropoiesis (production of red blood cells from precursors found in the bone marrow). The symptoms of anemia can include weakness, dizziness, fatigue, pallor, impairment of cognitive function and a general reduction in quality of life. Chronic and/or severe anemia can lead to the exacerbation of myocardial, cerebral or peripheral ischemia and to heart failure. Ischemia is defined as an absolute or relative shortage of oxygen to a tissue or organ and can result from disorders such as atherosclerosis, diabetes, thromboembolisms, hypotension, etc. The heart, brain and kidney are especially sensitive to ischemic stress caused by low blood supply.

The primary pharmacological treatment for anemia is administration of some variant of recombinant human erythropoietin (EPO). For anemias associated with kidney disease, chemotherapy-induced anemia, anemia from HIV-therapy or anemia due to blood loss, recombinant EPO is administered to enhance the supply of the hormone, correct the shortage of red blood cells and increase the blood's oxygen-carrying capacity. EPO replacement is not always sufficient to stimulate optimal erythropoiesis (e.g., in patients with iron processing deficiencies) and has associated risks.

Hypoxia-inducible factor (HIF) has been identified as a primary regulator of the cellular response to low oxygen. HIF is a heterodimeric gene transcription factor consisting of a highly regulated α-subunit (HIF-α) and a constitutively expressed β-subunit (HIF-β, also known as ARNT, or aryl hydrocarbon receptor nuclear transporter). HIF target genes are reported to be associated with various aspects of erythropoiesis (e.g., erythropoietin (EPO) and EPO receptor), glycolysis and angiogenesis (e.g., vascular endothelial growth factor (VEGF)). Genes for proteins involved in iron absorption, transport and utilization as well as heme synthesis are also targets of HIF.

Under normal oxygenation, HIF-α is a substrate in a reaction with molecular oxygen, which is catalyzed by a family of iron(II)-, 2-ketoglutarate- and ascorbate-dependent dioxygenase enzymes called PHD-1 (EGLN2, or egg laying abnormal 9 homolog 2, PHD2 (EGLN1), and PHD3 (EGLN3). Proline residues of HIF-α are hydroxylated (e.g., Pro-402 and Pro-564 of HIF-1α) and the resulting product is a target of the tumor suppressor protein von-Hippel Lindau, a component of an E3 ubiquitin ligase multiprotein complex involved in protein ubiquitination. Under low oxygenation, the HIF-α hydroxylation reaction is less efficient and HIF-α is available to dimerize with HIF-β. HIF dimers are translocated to the cell nucleus where they bind to a hypoxia-responsive enhancer element of HIF target genes.

Cellular levels of HIF are known to increase under conditions of hypoxia and after exposure to hypoxia mimetic agents. The latter includes, but is not limited to, specific metal ions (e.g., cobalt, nickel, manganese), iron chelators (e.g., desferrioxamine) and analogs of 2-ketoglurate (e.g., N-oxalyl glycine). The compounds of the present invention inhibit the HIF prolyl hydroxylases (PHD-1, PHD-2, PHD-3) and can also serve to modulate HIF levels. These compounds therefore have utility for the treatment and/or prevention of disorders or conditions where HIF modulation is desirable, such as anemia and ischemia. As an alternative to recombinant erythropoietin therapy, the compounds of the present invention provide a simpler and broader method for the management of anemia.

### SUMMARY OF THE INVENTION

The present invention concerns compounds of formula I

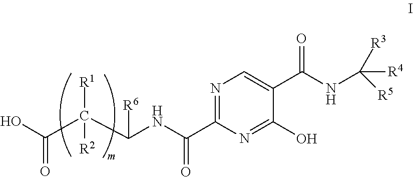

which inhibit HIF prolyl hydroxylase, their use for enhancing endogenous production of erythropoietin, and for treating conditions associated with reduced endogenous production of erythropoietin such as anemia and like conditions, as well as pharmaceutical compositions comprising such a compound and a pharmaceutical carrier.

### DETAILED DESCRIPTION OF THE INVENTION

The present invention provides compounds of formula I or stereoisomers or pharmaceutically acceptable salts thereof:

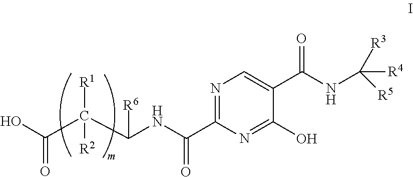

m is 0 or 1;
R¹ and R² are each independently selected from hydrogen, C₁₋₃alkyl, hydroxyC₁₋₃alkyl, and hydroxy, wherein R¹ and R² may optionally join together with the carbon to which they are attached to form a 3 to 7 membered saturated ring;
R³ is hydrogen, or C₁₋₃alkyl;
R⁴ and R⁵ are each independently selected from phenyl, C₁₋₃alkyl, quinolinyl, 2-3-dihydrobenzofuranyl, C₁₋₆haloalkyl, and pyridinyl, wherein R⁴ and R⁵ are each optionally substituted with 0, 1, or 2 R⁷;
further wherein R³ and R⁴ may optionally join together with the carbon to which they are attached to form a 3 to 7 membered saturated ring;
R⁶ is hydrogen arylC₀₋₅ alkyl, or heteroarylC₀₋₅ alkyl; and
R⁷ is selected from cyano, C₁₋₃alkoxy, halogen, C₁₋₆haloalkyl, phenyl, isoquinolinyl, pyridinyl, pyrazolyl, —NH(C₁₋₃alkyl), and phenoxy, wherein R⁷ is optionally substituted with 0, 1, or 2 C₁₋₃alkoxy, halogen, cyano, or C₁₋₆haloalkyl(oxy)₀₋₁.

Representative compounds of the instant invention include, but are not limited to, the following compounds and their pharmaceutically acceptable salts and their stereoisomers thereof:
(R)-3-(5-(Benzhydrylcarbamoyl)-4-hydroxypyrimidine-2-carboxamido)-2-methyl propanoic acid;
(S)-3-(5-(Benzhydrylcarbamoyl)-4-hydroxypyrimidine-2-carboxamido)-2-methyl propanoic acid;
3-(5-(Benzhydrylcarbamoyl)-4-hydroxypyrimidine-2-carboxamido)-2-hydroxy-2-methylpropanoic acid;
1-((5-(benzhydrylcarbamoyl)-4-hydroxypyrimidine-2-carboxamido)methyl)cyclopropanecarboxylic acid;
3-(5-(benzhydrylcarbamoyl)-4-hydroxypyrimidine-2-carboxamido)-2-hydroxypropanoic acid;
(2S)-2-(5-(benzhydrylcarbamoyl)-4-hydroxypyrimidine-2-carboxamido)-3-(3aH-indol-3-yl)propanoic acid;
(2R)-2-(5-(benzhydrylcarbamoyl)-4-hydroxypyrimidine-2-carboxamido)-3-(3aH-indol-3-yl)propanoic acid;
2-(5-(benzhydrylcarbamoyl)-4-hydroxypyrimidine-2-carboxamido)-3-(3aH-indol-3-yl)propanoic acid;
(S)-3-(5-(benzhydrylcarbamoyl)-4-hydroxypyrimidine-2-carboxamido)-2-hydroxypropanoic acid;
(R)-3-(5-(benzhydrylcarbamoyl)-4-hydroxypyrimidine-2-carboxamido)-2-hydroxypropanoic acid;
3-(5-(benzhydrylcarbamoyl)-4-hydroxypyropanoic acid;
(R)-3-(5-(((4-Cyanophenyl)(phenyl)methyl)carbamoyl)-4-hydroxypyrimidine-2-carboxamido)-2-hydroxypropanoic acid;
(S)-3-(5-(((4-Cyanophenyl)(phenyl)methyl)carbamoyl)-4-hydroxypyrimidine-2-carboxamido)-2-hydroxypropanoic acid;
3-(5-(((4-Cyanophenyl)(phenyl)methyl)carbamoyl)-4-hydroxypyrimidine-2-carboxamido)-2-hydroxypropanoic acid;
(R)-2-hydroxy-3-(4-hydroxy-5-(((S)-(4-methoxyphenyl) (phenyl)methyl)carbamoyl)pyrimidine-2-carboxamido)propanoic acid;
(R)-2-hydroxy-3-(4-hydroxy-5-((2-(4-(trifluoromethyl)phenyl)propan-2-yl)carbamoyl)pyrimidine-2-carboxamido)propanoic acid;
(S)-2-hydroxy-3-(4-hydroxy-5-((2-(4-(trifluoromethyl)phenyl)propan-2-yl)carbamoyl)pyrimidine-2-carboxamido)propanoic acid;
2-hydroxy-3-(4-hydroxy-5-((2-(4-(trifluoromethyl)phenyl)propan-2-yl)carbamoyl)pyrimidine-2-carboxamido)propanoic acid;
(R)-2-hydroxy-3-(4-hydroxy-5-(((R)-(4-methoxyphenyl) (phenyl)methyl)carbamoyl)pyrimidine-2-carboxamido)propanoic acid;
2-hydroxy-3-(4-hydroxy-5-(((4-methoxyphenyl) (phenyl)methyl)carbamoyl)pyrimidine-2-carboxamido)propanoic acid;
3-(5-((Bis(4-chlorophenyl)methyl)carbamoyl)-4-hydroxypyrimidine-2-carboxamido)-2-methylpropanoic acid;
3-(4-hydroxy-5-(((4-methoxyphenyl)(6-methoxyquinolin-2-yl)methyl)carbamoyl)pyrimidine-2-carboxamido)-2-methylpropanoic acid;
(R)-3-(4-hydroxy-5-(((4-methoxyphenyl)(6-methoxyquinolin-2-yl)methyl)carbamoyl)pyrimidine-2-carboxamido)-2-methylpropanoic acid;
(S)-3-(4-hydroxy-5-(((4-methoxyphenyl)(6-methoxyquinolin-2-yl)methyl)carbamoyl)pyrimidine-2-carboxamido)-2-methylpropanoic acid;
3-(5-((bis(4-methoxyphenyl)methyl)carbamoyl)-4-hydroxypyrimidine-2-carboxamido)-2-methylpropanoic acid;
(R)-3-(5-((bis(4-methoxyphenyl)methyl)carbamoyl)-4-hydroxypyrimidine-2-carboxamido)-2-methylpropanoic acid;
(S)-3-(5-((bis(4-methoxyphenyl)methyl)carbamoyl)-4-hydroxypyrimidine-2-carboxamido)-2-methylpropanoic acid;
3-(5-((1-(4-bromophenyl)cyclopropyl)carbamoyl)-4-hydroxypyrimidine-2-carboxamido)-2-methylpropanoic acid;
(S)-3-(5-((1-(4-bromophenyl)cyclopropyl)carbamoyl)-4-hydroxypyrimidine-2-carboxamido)-2-methylpropanoic acid;
(R)-3-(5-((1-(4-bromophenyl)cyclopropyl)carbamoyl)-4-hydroxypyrimidine-2-carboxamido)-2-methylpropanoic acid;
3-(5-((bis(4-methoxyphenyl)methyl)carbamoyl)-4-hydroxypyrimidine-2-carboxamido)-2-methylpropanoic acid;
(S)-3-(5-((bis(4-methoxyphenyl)methyl)carbamoyl)-4-hydroxypyrimidine-2-carboxamido)-2-methylpropanoic acid;
(R)-3-(5-((bis(4-methoxyphenyl)methyl)carbamoyl)-4-hydroxypyrimidine-2-carboxamido)-2-methylpropanoic acid;
3-(4-hydroxy-5-((2-(6-(4-methoxyphenyl)pyridin-3-yl)propan-2-yl)carbamoyl)pyrimidine-2-carboxamido)-2-methylpropanoic acid;
(R)-3-(4-hydroxy-5-((2-(6-(4-methoxyphenyl)pyridin-3-yl)propan-2-yl)carbamoyl)pyrimidine-2-carboxamido)-2-methylpropanoic acid;
(S)-3-(4-hydroxy-5-((2-(6-(4-methoxyphenyl)pyridin-3-yl)propan-2-yl)carbamoyl)pyrimidine-2-carboxamido)-2-methylpropanoic acid;
3-(4-hydroxy-5-((2-(4′-(trifluoromethyl)-[1,1′-biphenyl]-4-yl)propan-2-yl)carbamoyl)pyrimidine-2-carboxamido)-2-methylpropanoic acid;
2-((2-carboxypropyl)carbamoyl)-6-hydroxy-5-((2-(4-(isoquinolin-5-yl)phenyl)propan-2-yl)carbamoyl)pyrimidin-1-ium formate;
(S)-2-((2-carboxypropyl)carbamoyl)-6-hydroxy-5-((2-(4-(isoquinolin-5-yl)phenyl)propan-2-yl)carbamoyl)pyrimidin-1-ium formate;
(R)-2-((2-carboxypropyl)carbamoyl)-6-hydroxy-5-((2-(4-(isoquinolin-5-yl)phenyl) propan-2-yl)carbamoyl)pyrimidin-1-ium formate;
2-((2-carboxypropyl)carbamoyl)-6-hydroxy-5-((2-(4-(pyridin-3-yl)phenyl)propan-2-yl)carbamoyl)pyrimidin-1-ium formate;
(R)-2-((2-carboxypropyl)carbamoyl)-6-hydroxy-5-((2-(4-(pyridin-3-yl)phenyl)propan-2-yl)carbamoyl)pyrimidin-1-ium formate;
(S)-2-((2-carboxypropyl)carbamoyl)-6-hydroxy-5-((2-(4-(pyridin-3-yl)phenyl)propan-2-yl)carbamoyl)pyrimidin-1-ium formate;
3-(5-((2(4′-cyano-2′-methyl-[1,1′-biphenyl]-4-yl)propan-2-yl)carbamoyl)-4-hydroxypyrimidine-2-carboxamido)-2-methylpropanoic acid;
(R)-3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
(S)-3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
3-(5-((2-(4′-fluoro-[1,1′-biphenyl]-4-yl)propan-2-yl)carbamoyl)-4-hydroxypyrimidine-2-carboxamido)-2-methylpropanoic acid;
(R)-3-(5-((2-(4′-fluoro-[1,1′-biphenyl]-4-yl)propan-2-yl)carbamoyl)-4-hydroxypyrimidine-2-carboxamido)-2-methylpropanoic acid;
(S)-3-(5-((2-(4′-fluoro-[1,1′-biphenyl]-4-yl)propan-2-yl)carbamoyl)-4-hydroxypyrimidine-2-carboxamido)-2-methylpropanoic acid;
3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(R)-3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(S)-3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(R)-2-((2-carboxypropyl)carbamoyl)-6-hydroxy-5-((2-(4-(6-methoxypyridin-3-yl)phenyl)propan-2-yl)carbamoyl)pyrimidin-1-ium chloride;
2-((2-carboxypropyl)carbamoyl)-6-hydroxy-5-((2-(4-(6-methoxypyridin-3-yl)phenyl)propan-2-yl)carbamoyl)pyrimidin-1-ium chloride;
(S)-2-((2-carboxypropyl)carbamoyl)-6-hydroxy-5-((2-(4-(6-methoxypyridin-3-yl)phenyl)propan-2-yl)carbamoyl)pyrimidin-1-ium chloride;
5-((2-(4-(1H-pyrazol-3-yl)phenyl)propan-2-yl)carbamoyl)-2-((2-carboxypropyl)carbamoyl)-6-hydroxypyrimidin-1-ium formate;
(R)-5-((2-(4-(1H-pyrazol-3-yl)phenyl)propan-2-yl)carbamoyl)-2-((2-carboxypropyl)carbamoyl)-6-hydroxypyrimidin-1-ium formate; (S)-5-((2-(4-(1H-pyrazol-3-yl)phenyl)propan-2-yl)carbamoyl)-2-((2-carboxypropyl)carbamoyl)-6-hydroxypyrimidin-1-ium formate;
3-(5-((2-(4′-chloro-[1,1′-biphenyl]-4-yl)propan-2-yl)carbamoyl)-4-hydroxypyrimidine-2-carboxamido)-2-methylpropanoic acid;
(R)-3-(5-((2-(4′-chloro-[1,1′-biphenyl]-4-yl)propan-2-yl)carbamoyl)-4-hydroxypyrimidine-2-carboxamido)-2-methylpropanoic acid;
(S)-3-(5-((2-(4′-chloro-[1,1′-biphenyl]-4-yl)propan-2-yl)carbamoyl)-4-hydroxypyrimidine-2-carboxamido)-2-methylpropanoic acid;
3-(4-hydroxy-5-((1-(4′-(trifluoromethyl)-[1,1′-biphenyl]-4-yl)cyclopropyl)carbamoyl)pyrimidine-2-carboxamido)-2-methyl propanoic acid;
(R)-3-(4-hydroxy-5-((1-(4′-(trifluoromethyl)-[1,1′-biphenyl]-4-yl)cyclopropyl)carbamoyl)pyrimidine-2-carboxamido)-2-methyl propanoic acid;
(S)-3-(4-hydroxy-5-((1-(4′-(trifluoromethyl)-[1,1′-biphenyl]-4-yl)cyclopropyl)carbamoyl)pyrimidine-2-carboxamido)-2-methyl propanoic acid;
3-(5-((1-(4′-chloro-[1,1′-biphenyl]-4-yl)cyclopropyl)carbamoyl)-4-hydroxypyrimidine-2-carboxamido)-2-methylpropanoic acid;
(R)-3-(5-((1-(4′-chloro-[1,1′-biphenyl]-4-yl)cyclopropyl)carbamoyl)-4-hydroxypyrimidine-2-carboxamido)-2-methylpropanoic acid;
(S)-3-(5-((1-(4′-chloro-[1,1′-biphenyl]-4-yl)cyclopropyl)carbamoyl)-4-hydroxypyrimidine-2-carboxamido)-2-methylpropanoic acid;
2-((2-carboxypropyl)carbamoyl)-6-hydroxy-5-((1-(4-(isoquinolin-5-yl)phenyl)cyclopropyl)carbamoyl)pyrimidin-1-ium formate;
(S)-2-((2-carboxypropyl)carbamoyl)-6-hydroxy-5-((1-(4-(isoquinolin-5-yl)phenyl)cyclopropyl)carbamoyl)pyrimidin-1-ium formate;
(R)-2-((2-carboxypropyl)carbamoyl)-6-hydroxy-5-((1-(4-(isoquinolin-5-yl)phenyl)cyclopropyl)carbamoyl)pyrimidin-1-ium formate;
3-(5-((1-(4′-fluoro-[1,1′-biphenyl]-4-yl)cyclopropyl)carbamoyl)-4-hydroxypyrimidine-2-carboxamido)-2-methylpropanoic acid;
(R)-3-(5-((1-(4′-fluoro-[1,1′-biphenyl]-4-yl)cyclopropyl)carbamoyl)-4-hydroxypyrimidine-2-carboxamido)-2-methylpropanoic acid;
(S)-3-(5-((1-(4′-fluoro-[1,1′-biphenyl]-4-yl)cyclopropyl)carbamoyl)-4-hydroxypyrimidine-2-carboxamido)-2-methylpropanoic acid;
2-((2-carboxypropyl)carbamoyl)-6-hydroxy-5-((1-(4-(6-methoxypyridin-3-yl)phenyl)cyclopropyl)carbamoyl)pyrimidin-1-ium chloride;
(R)-2-((2-carboxypropyl)carbamoyl)-6-hydroxy-5-((1-(4-(6-methoxypyridin-3-yl)phenyl)cyclopropyl)carbamoyl)pyrimidin-1-ium chloride;
(S)-2-((2-carboxypropyl)carbamoyl)-6-hydroxy-5-((1-(4-(6-methoxypyridin-3-yl)phenyl)cyclopropyl)carbamoyl)pyrimidin-1-ium chloride; 3-(4-hydroxy-5-(((4-methoxyphenyl)(6-(methylamino)pyridin-3-yl)methyl)carbamoyl)pyrimidine-2-carboxamido)propanoic acid;
(R)-3-(4-hydroxy-5-(((4-methoxyphenyl)(6-(methylamino)pyridin-3-yl)methyl)carbamoyl)pyrimidine-2-carboxamido)propanoic acid;
(S)-3-(4-hydroxy-5-(((4-methoxyphenyl)(6-(methylamino)pyridin-3-yl)methyl)carbamoyl)pyrimidine-2-carboxamido)propanoic acid;
3-(4-hydroxy-5-(((4-methoxyphenyl) (phenyl)methyl)carbamoyl)pyrimidine-2-carboxamido)propanoic acid;
(S)-3-(4-hydroxy-5-(((4-methoxyphenyl)(phenyl)methyl)carbamoyl)pyrimidine-2-carboxamido)propanoic acid;
(S)-3-(5-(((4-chlorophenyl)(4-methoxyphenyl)methyl)carbamoyl)-4-hydroxypyrimidine-2-carboxamido)propanoic acid;
(R)-3-(5-(((4-chlorophenyl)(4-methoxyphenyl)methyl)carbamoyl)-4-hydroxypyrimidine-2-carboxamido)propanoic acid;
3-(5-(((4-chlorophenyl)(4-methoxyphenyl)methyl)carbamoyl)-4-hydroxypyrimidine-2-carboxamido)propanoic acid;
3-(5-(((2,3-dihydrobenzofuran-5-yl)(4-methoxyphenyl)methyl)carbamoyl)-4-hydroxypyrimidine-2-carboxamido)propanoic acid;
3-(5-(((4-chlorophenyl)(4-(trifluoromethyl)phenyl)methyl)carbamoyl)-4-hydroxypyrimidine-2-carboxamido)propanoic acid
3-(5-((bis(6-methoxypyridin-3-yl)methyl)carbamoyl)-4-hydroxypyrimidine-2-carboxamido)propanoic acid;
3-(5-((bis(4-chlorophenyl)methyl)carbamoyl)-4-hydroxypyrimidine-2-carboxamido)propanoic acid;
(R)-N5-(1-(4-bromophenyl)ethyl)-4-hydroxy-N2-propylpyrimidine-2,5-dicarboxamide;
N5-(1-(4-bromophenyl)ethyl)-4-hydroxy-N2-propylpyrimidine-2,5-dicarboxamide;
3-(5-((1-(4′-fluoro-[1,1′-biphenyl]-4-yl)ethyl)carbamoyl)-4-hydroxypyrimidine-2-carboxamido)propanoic acid;
3-(5-((1-(4-bromophenyl)cyclopropyl)carbamoyl)-4-hydroxypyrimidine-2-carboxamido)propanoic acid;
3-(4-hydroxy-5-((1-(4-phenoxyphenyl)ethyl)carbamoyl)pyrimidine-2-carboxamido)propanoic acid;
3-(5-((1-(4-bromophenyl)-2,2,2-trifluoroethyl)carbamoyl)-4-hydroxypyrimidine-2-carboxamido) propanoic acid;
3-(5-((bis(4-methoxyphenyl)methyl)carbamoyl)-4-hydroxypyrimidine-2-carboxamido)propanoic acid; and
3-(4-hydroxy-5-((phenyl(4-(trifluoromethyl)phenyl)methyl)carbamoyl)pyrimidine-2-carboxamido) propanoic acid.

In one embodiment of the invention, R¹ and R² are each independently selected from hydrogen, C₁₋₃alkyl, and hydroxy, wherein R¹ and R² may optionally join together with the carbon to which they are attached to form a 3 to 7 membered saturated ring.

In another embodiment of the invention, R¹ is hydrogen or methyl and R² is selected from hydrogen, methyl and hydroxy, wherein R¹ and R² may optionally join together with the carbon to which they are attached to form a cyclopropyl ring. In a variant of this invention, R¹ is hydrogen and R² is selected from hydrogen, methyl and hydroxy.

In one embodiment of the invention, R⁶ is hydrogen or heteroarylC₀₋₅ alkyl. In a variant of this embodiment, R⁶ is hydrogen or indolylmethyl. In another variant of this embodiment, R⁶ is hydrogen. In yet another variant, R⁶ is indolylmethyl.

In an embodiment, R⁴ is selected from phenyl, C₁₋₃alkyl, C₁₋₆haloalkyl, and pyridinyl, and R⁵ is selected from selected from phenyl, C₁₋₃alkyl, quinolinyl, 2,3-dihydrobenzofuranyl, and pyridinyl, wherein R⁴ and R⁵ are each optionally substituted with 0, 1, or 2 R⁷.

In yet another embodiment, R³ is hydrogen or methyl.

In one embodiment of the invention, R³ and R⁴ join together with the carbon to which they are attached to form a 3 to 6 membered saturated ring. In a variant of this embodiment, R³ and R⁴ join together with the carbon to which they are attached to form a ring selected from cyclopropyl, cyclobutyl, cyclopentyl and cyclohexyl. In another variant, R³ and R⁴ join together with the carbon to which they are attached to form a cyclopropyl ring.

In one embodiment of the invention, R⁷ is selected from cyano, methoxy, ethoxy, halogen, trifluoromethyl, trifluoroethyl, difluoromethyl, difluoroethyl, phenyl, isoquinolinyl, pyridinyl, pyrazolyl, methylamino, ethylamino, and phenoxy, wherein R⁷ is optionally substituted with 0, 1, or 2 C₁₋₃alkoxy, halogen, cyano, or C₁₋₆haloalkyl(oxy)₀₋₁.

In one embodiment of the invention, R⁷ is selected from cyano, methoxy, halogen, trifluoromethyl, phenyl, isoquinolinyl, pyridinyl, pyrazolyl, methylamino, phenoxy, wherein R⁷ is optionally substituted with 0, 1, or 2 methoxy, halogen, cyano, trifluoromethyl or trifluoromethoxy.

In a particular embodiment of the invention, are compounds of formula I or stereoisomers or pharmaceutically acceptable salts thereof:

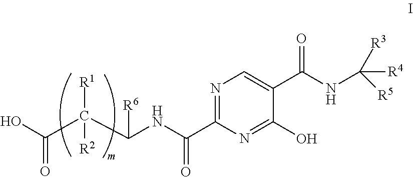

m is 0 or 1;
R¹ is hydrogen or methyl;
R² is selected from hydrogen, methyl and hydroxy,
wherein R¹ and R² may optionally join together with the carbon to which they are attached to form a cyclopropyl ring.
R³ is hydrogen, or methyl;
R⁴ is selected from phenyl, methyl, trifluoromethyl, and pyridinyl; and
R⁵ is selected from selected from phenyl, C₁₋₃alkyl, quinolinyl, 2,3-dihydrobenzofuranyl, and pyridinyl, wherein R⁴ and R⁵ are each optionally substituted with 0, 1, or 2 R⁷;
further wherein R³ and R⁴ may optionally join together with the carbon to which they are attached to form a ring selected from a cyclopropyl ring;
R⁶ is hydrogen or indolylmethyl; and
R⁷ is selected from cyano, methyl, halogen, trifluoromethyl, phenyl, isoquinolinyl, pyridinyl, pyrazolyl, methylamino, phenoxy, wherein R⁷ is optionally substituted with 0, 1, or 2 methoxy, halogen, cyano, trifluoromethyl, or trifluoromethoxy.

One embodiment of the invention includes compounds of the instant invention and their pharmaceutically acceptable salts and their stereoisomers thereof:
(R)-3-(5-(Benzhydrylcarbamoyl)-4-hydroxypyrimidine-2-carboxamido)-2-methyl propanoic acid;
(S)-3-(5-(Benzhydrylcarbamoyl)-4-hydroxypyrimidine-2-carboxamido)-2-methyl propanoic acid;
3-(5-(Benzhydrylcarbamoyl)-4-hydroxypyrimidine-2-carboxamido)-2-hydroxy-2-methylpropanoic acid;
1-((5-(benzhydrylcarbamoyl)-4-hydroxypyrimidine-2-carboxamido)methyl)cyclopropanecarboxylic acid;
3-(5-(benzhydrylcarbamoyl)-4-hydroxypyrimidine-2-carboxamido)-2-hydroxypropanoic acid; (S)-3-(5-(benzhydrylcarbamoyl)-4-hydroxypyrimidine-2-carboxamido)-2-hydroxypropanoic acid;
(R)-3-(5-(benzhydrylcarbamoyl)-4-hydroxypyrimidine-2-carboxamido)-2-hydroxypropanoic acid;
3-(5-(benzhydrylcarbamoyl)-4-hydroxypyrimidine-2-carboxamido)-2-hydroxypropanoic acid;
(R)-3-(5-(((4-Cyanophenyl)(phenyl)methyl)carbamoyl)-4-hydroxypyrimidine-2-carboxamido)-2-hydroxypropanoic acid;
(S)-3-(5-(((4-Cyanophenyl)(phenyl)methyl)carbamoyl)-4-hydroxypyrimidine-2-carboxamido)-2-hydroxypropanoic acid;
3-(5-(((4-Cyanophenyl)(phenyl)methyl)carbamoyl)-4-hydroxypyrimidine-2-carboxamido)-2-hydroxypropanoic acid;
(R)-2-hydroxy-3-(4-hydroxy-5-(((S)-(4-methoxyphenyl) (phenyl)methyl)carbamoyl)pyrimidine-2-carboxamido)propanoic acid;
(R)-2-hydroxy-3-(4-hydroxy-5-((2-(4-(trifluoromethyl)phenyl)propan-2-yl)carbamoyl)pyrimidine-2-carboxamido)propanoic acid;
(S)-2-hydroxy-3-(4-hydroxy-5-((2-(4-(trifluoromethyl)phenyl)propan-2-yl)carbamoyl)pyrimidine-2-carboxamido)propanoic acid;
2-hydroxy-3-(4-hydroxy-5-((2-(4-(trifluoromethyl)phenyl)propan-2-yl)carbamoyl)pyrimidine-2-carboxamido)propanoic acid;
(R)-2-hydroxy-3-(4-hydroxy-5-(((R)-(4-methoxyphenyl) (phenyl)methyl)carbamoyl)pyrimidine-2-carboxamido)propanoic acid;
2-hydroxy-3-(4-hydroxy-5-(((4-methoxyphenyl) (phenyl)methyl)carbamoyl)pyrimidine-2-carboxamido)propanoic acid;
3-(5-((Bis(4-chlorophenyl)methyl)carbamoyl)-4-hydroxypyrimidine-2-carboxamido)-2-methylpropanoic acid;
3-(4-hydroxy-5-(((4-methoxyphenyl)(6-methoxyquinolin-2-yl)methyl)carbamoyl)pyrimidine-2-carboxamido)-2-methylpropanoic acid;
(R)-3-(4-hydroxy-5-(((4-methoxyphenyl)(6-methoxyquinolin-2-yl)methyl)carbamoyl)pyrimidine-2-carboxamido)-2-methylpropanoic acid;
(S)-3-(4-hydroxy-5-(((4-methoxyphenyl)(6-methoxyquinolin-2-yl)methyl)carbamoyl)pyrimidine-2-carboxamido)-2-methylpropanoic acid;
3-(5-((bis(4-methoxyphenyl)methyl)carbamoyl)-4-hydroxypyrimidine-2-carboxamido)-2-methylpropanoic acid;
(R)-3-(5-((bis(4-methoxyphenyl)methyl)carbamoyl)-4-hydroxypyrimidine-2-carboxamido)-2-methylpropanoic acid;
(S)-3-(5-((bis(4-methoxyphenyl)methyl)carbamoyl)-4-hydroxypyrimidine-2-carboxamido)-2-methylpropanoic acid;
3-(5-((1-(4-bromophenyl)cyclopropyl)carbamoyl)-4-hydroxypyrimidine-2-carboxamido)-2-methylpropanoic acid;
(S)-3-(5-((1-(4-bromophenyl)cyclopropyl)carbamoyl)-4-hydroxypyrimidine-2-carboxamido)-2-methylpropanoic acid;
(R)-3-(5-((1-(4-bromophenyl)cyclopropyl)carbamoyl)-4-hydroxypyrimidine-2-carboxamido)-2-methylpropanoic acid;
3-(5-((bis(4-methoxyphenyl)methyl)carbamoyl)-4-hydroxypyrimidine-2-carboxamido)-2-methylpropanoic acid;
(S)-3-(5-((bis(4-methoxyphenyl)methyl)carbamoyl)-4-hydroxypyrimidine-2-carboxamido)-2-methylpropanoic acid;
(R)-3-(5-((bis(4-methoxyphenyl)methyl)carbamoyl)-4-hydroxypyrimidine-2-carboxamido)-2-methylpropanoic acid;
3-(4-hydroxy-5-((2-(6-(4-methoxyphenyl)pyridin-3-yl)propan-2-yl)carbamoyl)pyrimidine-2-carboxamido)-2-methylpropanoic acid;
(R)-3-(4-hydroxy-5-((2-(6-(4-methoxyphenyl)pyridin-3-yl)propan-2-yl)carbamoyl)pyrimidine-2-carboxamido)-2-methylpropanoic acid;
(S)-3-(4-hydroxy-5-((2-(6-(4-methoxyphenyl)pyridin-3-yl)propan-2-yl)carbamoyl)pyrimidine-2-carboxamido)-2-methylpropanoic acid;
3-(4-hydroxy-5-((2-(4′-(trifluoromethyl)-[1,1′-biphenyl]-4-yl)propan-2-yl)carbamoyl)pyrimidine-2-carboxamido)-2-methylpropanoic acid;
2-((2-carboxypropyl)carbamoyl)-6-hydroxy-5-((2-(4-(isoquinolin-5-yl)phenyl)propan-2-yl)carbamoyl)pyrimidin-1-ium formate;
(S)-2-((2-carboxypropyl)carbamoyl)-6-hydroxy-5-((2-(4-(isoquinolin-5-yl)phenyl)propan-2-yl)carbamoyl)pyrimidin-1-ium formate;
(R)-2-((2-carboxypropyl)carbamoyl)-6-hydroxy-5-((2-(4-(isoquinolin-5-yl)phenyl)propan-2-yl)carbamoyl)pyrimidin-1-ium formate;
2-((2-carboxypropyl)carbamoyl)-6-hydroxy-5-((2-(4-(pyridin-3-yl)phenyl)propan-2-yl)carbamoyl)pyrimidin-1-ium formate;
(R)-2-((2-carboxypropyl)carbamoyl)-6-hydroxy-5-((2-(4-(pyridin-3-yl)phenyl)propan-2-yl)carbamoyl)pyrimidin-1-ium formate;
(S)-2-((2-carboxypropyl)carbamoyl)-6-hydroxy-5-((2-(4-(pyridin-3-yl)phenyl)propan-2-yl)carbamoyl)pyrimidin-1-ium formate;
3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
(R)-3-(5-((2-(4′-cyano-2′-methyl-[, 1-biphenyl]-4-yl)propan-2-yl)carbamoyl)-4-hydroxypyrimidine-2-carboxamido)-2-methylpropanoic acid;
(S)-3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
3-(5-((2-(4′-fluoro-[1,1′-biphenyl]-4-yl)propan-2-yl)carbamoyl)-4-hydroxypyrimidine-2-carboxamido)-2-methylpropanoic acid;
(R)-3-(5-((2-(4′-fluoro-[1,1′-biphenyl]-4-yl)propan-2-yl)carbamoyl)-4-hydroxypyrimidine-2-carboxamido)-2-methylpropanoic acid;
(S)-3-(5-((2-(4′-fluoro-[1,1′-biphenyl]-4-yl)propan-2-yl)carbamoyl)-4-hydroxypyrimidine-2-carboxamido)-2-methylpropanoic acid;
3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(R)-3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(S)-3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(R)-2-((2-carboxypropyl)carbamoyl)-6-hydroxy-5-((2-(4-(6-methoxypyridin-3-yl)phenyl)propan-2-yl)carbamoyl)pyrimidin-1-ium chloride;
2-((2-carboxypropyl)carbamoyl)-6-hydroxy-5-((2-(4-(6-methoxypyridin-3-yl)phenyl)propan-2-yl)carbamoyl)pyrimidin-1-ium chloride;
(S)-2-((2-carboxypropyl)carbamoyl)-6-hydroxy-5-((2-(4-(6-methoxypyridin-3-yl)phenyl)propan-2-yl)carbamoyl)pyrimidin-1-ium chloride;
5-((2-(4-(1H-pyrazol-3-yl)phenyl)propan-2-yl)carbamoyl)-2-((2-carboxypropyl)carbamoyl)-6-hydroxypyrimidin-1-ium formate;
(R)-5-((2-(4-(1H-pyrazol-3-yl)phenyl)propan-2-yl)carbamoyl)-2-((2-carboxypropyl)carbamoyl)-6-hydroxypyrimidin-1-ium formate;
(S)-5-((2-(4-(1H-pyrazol-3-yl)phenyl)propan-2-yl)carbamoyl)-2-((2-carboxypropyl)carbamoyl)-6-hydroxypyrimidin-1-ium formate;
3-(5-((2-(4′-chloro-[1,1′-biphenyl]-4-yl)propan-2-yl)carbamoyl)-4-hydroxypyrimidine-2-carboxamido)-2-methylpropanoic acid;
(R)-3-(5-((2-(4′-chloro-[1,1′-biphenyl]-4-yl)propan-2-yl)carbamoyl)-4-hydroxypyrimidine-2-carboxamido)-2-methylpropanoic acid;
(S)-3-(5-((2-(4′-chloro-[1,1′-biphenyl]-4-yl)propan-2-yl)carbamoyl)-4-hydroxypyrimidine-2-carboxamido)-2-methylpropanoic acid;
3-(4-hydroxy-5-((1-(4′-(trifluoromethyl)-[1,1′-biphenyl]-4-yl)cyclopropyl) carbamoyl)pyrimidine-2-carboxamido)-2-methyl propanoic acid;
(R)-3-(4-hydroxy-5-((1-(4′-(trifluoromethyl)-[1,1′-biphenyl]-4-yl)cyclopropyl) carbamoyl)pyrimidine-2-carboxamido)-2-methyl propanoic acid;
(S)-3-(4-hydroxy-5-((1-(4′-(trifluoromethyl)-[1,1′-biphenyl]-4-yl)cyclopropyl) carbamoyl)pyrimidine-2-carboxamido)-2-methyl propanoic acid;
3-(5-((1-(4′-chloro-[1,1′-biphenyl]-4-yl)cyclopropyl)carbamoyl)-4-hydroxypyrimidine-2-carboxamido)-2-methylpropanoic acid;
(R)-3-(5-((1-(4′-chloro-[1,1′-biphenyl]-4-yl)cyclopropyl)carbamoyl)-4-hydroxypyrimidine-2-carboxamido)-2-methylpropanoic acid;
(S)-3-(5-((1-(4′-chloro-[1,1′-biphenyl]-4-yl)cyclopropyl)carbamoyl)-4-hydroxypyrimidine-2-carboxamido)-2-methylpropanoic acid;
2-((2-carboxypropyl)carbamoyl)-6-hydroxy-5-((1-(4-(isoquinolin-5-yl)phenyl)cyclopropyl)carbamoyl)pyrimidin-1-ium formate;
(S)-2-((2-carboxypropyl)carbamoyl)-6-hydroxy-5-((1-(4-(isoquinolin-5-yl)phenyl)cyclopropyl)carbamoyl)pyrimidin-1-ium formate;
(R)-2-((2-carboxypropyl)carbamoyl)-6-hydroxy-5-((1-(4-(isoquinolin-5-yl)phenyl)cyclopropyl)carbamoyl)pyrimidin-1-ium formate;
3-(5-((1-(4′-fluoro-[1,1′-biphenyl]-4-yl)cyclopropyl)carbamoyl)-4-hydroxypyrimidine-2-carboxamido)-2-methylpropanoic acid;
(R)-3-(5-((1-(4′-fluoro-[1,1′-biphenyl]-4-yl)cyclopropyl)carbamoyl)-4-hydroxypyrimidine-2-carboxamido)-2-methylpropanoic acid;
(S)-3-(5-((1-(4′-fluoro-[1,1′-biphenyl]-4-yl)cyclopropyl)carbamoyl)-4-hydroxypyrimidine-2-carboxamido)-2-methylpropanoic acid;
2-((2-carboxypropyl)carbamoyl)-6-hydroxy-5-((1-(4-(6-methoxypyridin-3-yl)phenyl)cyclopropyl)carbamoyl)pyrimidin-1-ium chloride;
(R)-2-((2-carboxypropyl)carbamoyl)-6-hydroxy-5-((1-(4-(6-methoxypyridin-3-yl)phenyl)cyclopropyl)carbamoyl)pyrimidin-1-ium chloride; and
(S)-2-((2-carboxypropyl)carbamoyl)-6-hydroxy-5-((1-(4-(6-methoxypyridin-3-yl)phenyl)cyclopropyl)carbamoyl)pyrimidin-1-ium chloride.

In another embodiment of the invention are following compounds and their pharmaceutically acceptable salts and their stereoisomers thereof:
(2S)-2-(5-(benzhydrylcarbamoyl)-4-hydroxypyrimidine-2-carboxamido)-3-(3aH-indol-3-yl)propanoic acid;
(2R)-2-(5-(benzhydrylcarbamoyl)-4-hydroxypyrimidine-2-carboxamido)-3-(3aH-indol-3-yl)propanoic acid;
2-(5-(benzhydrylcarbamoyl)-4-hydroxypyrimidine-2-carboxamido)-3-(3aH-indol-3-yl)propanoic acid;
3-(4-hydroxy-5-(((4-methoxyphenyl)(6-(methylamino)pyridin-3-yl)methyl)carbamoyl)pyrimidine-2-carboxamido)propanoic acid;
(R)-3-(4-hydroxy-5-(((4-methoxyphenyl)(6-(methylamino)pyridin-3-yl)methyl)carbamoyl)pyrimidine-2-carboxamido)propanoic acid;
(S)-3-(4-hydroxy-5-(((4-methoxyphenyl)(6-(methylamino)pyridin-3-yl)methyl)carbamoyl)pyrimidine-2-carboxamido)propanoic acid;
3-(4-hydroxy-5-(((4-methoxyphenyl)(phenyl)methyl)carbamoyl)pyrimidine-2-carboxamido)propanoic acid;
(S)-3-(4-hydroxy-5-(((4-methoxyphenyl)(phenyl)methyl)carbamoyl)pyrimidine-2-carboxamido)propanoic acid;
(S)-3-(5-(((4-chlorophenyl)(4-methoxyphenyl)methyl)carbamoyl)-4-hydroxypyrimidine-2-carboxamido)propanoic acid;
(R)-3-(5-(((4-chlorophenyl)(4-methoxyphenyl)methyl)carbamoyl)-4-hydroxypyrimidine-2-carboxamido)propanoic acid;
3-(5-(((4-chlorophenyl)(4-methoxyphenyl)methyl)carbamoyl)-4-hydroxypyrimidine-2-carboxamido)propanoic acid;
3-(5-(((2,3-dihydrobenzofuran-5-yl)(4-methoxyphenyl)methyl)carbamoyl)-4-hydroxypyrimidine-2-carboxamido)propanoic acid;
3-(5-(((4-chlorophenyl)(4-(trifluoromethyl)phenyl)methyl)carbamoyl)-4-hydroxypyrimidine-2-carboxamido)propanoic acid
3-(5-((bis(6-methoxypyridin-3-yl)methyl)carbamoyl)-4-hydroxypyrimidine-2-carboxamido)propanoic acid;
3-(5-((bis(4-chlorophenyl)methyl)carbamoyl)-4-hydroxypyrimidine-2-carboxamido)propanoic acid;
(R)-N5-(1-(4-bromophenyl)ethyl)-4-hydroxy-N2-propylpyrimidine-2,5-dicarboxamide;
N5-(1-(4-bromophenyl)ethyl)-4-hydroxy-N2-propylpyrimidine-2,5-dicarboxamide;
3-(5-((1-(4′-fluoro-[1,1′-biphenyl]-4-yl)ethyl)carbamoyl)-4-hydroxypyrimidine-2-carboxamido)propanoic acid;
3-(5-((1-(4-bromophenyl)cyclopropyl)carbamoyl)-4-hydroxypyrimidine-2-carboxamido)propanoic acid;
3-(4-hydroxy-5-((1-(4-phenoxyphenyl)ethyl)carbamoyl)pyrimidine-2-carboxamido)propanoic acid;
3-(5-((1-(4-bromophenyl)-2,2,2-trifluoroethyl)carbamoyl)-4-hydroxypyrimidine-2-carboxamido) propanoic acid;
3-(5-((bis(4-methoxyphenyl)methyl)carbamoyl)-4-hydroxypyrimidine-2-carboxamido)propanoic acid; and
3-(4-hydroxy-5-((phenyl(4-(trifluoromethyl)phenyl)methyl)carbamoyl)pyrimidine-2-carboxamido) propanoic acid.

As used herein except where noted, “alkyl” is intended to include both branched- and straight-chain saturated aliphatic hydrocarbon groups, including all isomers, having the specified number of carbon atoms. Commonly used abbreviations for alkyl groups are used throughout the specification, e.g. methyl may be represented by “Me” or CH₃, ethyl may be represented by “Et” or CH₂CH₃, propyl may be represented by “Pr” or CH₂CH₂CH₃, butyl may be represented by “Bu” or CH₂CH₂CH₂CH₃, etc. “C₁₋₆ alkyl” (or “C₁-C₆ alkyl”) for example, means linear or branched chain alkyl groups, including all isomers, having the specified number of carbon atoms. C₁₋₆ alkyl includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl.

The term “halogen” (or “halo”) refers to fluorine, chlorine, bromine and iodine (alternatively referred to as fluoro (F), chloro (Cl), bromo (Br), and iodo (I)).

The term “aryl” refers to aromatic mono- and poly-carbocyclic ring systems, wherein the individual carbocyclic rings in the polyring systems are fused or attached to each other via a single bond. Suitable aryl groups include phenyl, naphthyl, and biphenylenyl.

The term “carbocycle” (and variations thereof such as “carbocyclic” or “carbocyclyl”) as used herein, unless otherwise indicated, refers to (i) a C₃ to C₈ monocyclic, saturated or unsaturated ring or (ii) a C₇ to C₁₂ bicyclic saturated or unsaturated ring system. Each ring in (ii) is either independent of, or fused to, the other ring, and each ring is saturated or unsaturated. The carbocycle may be attached to the rest of the molecule at any carbon atom which results in a stable compound. The fused bicyclic carbocycles are a subset of the carbocycles; i.e., the term “fused bicyclic carbocycle” generally refers to a C₇ to C₁₀ bicyclic ring system in which each ring is saturated or unsaturated and two adjacent carbon atoms are shared by each of the rings in the ring system. A fused bicyclic carbocycle in which one ring is saturated and the other is saturated is a saturated bicyclic ring system. A fused bicyclic carbocycle in which one ring is benzene and the other is saturated is an unsaturated bicyclic ring system. A fused bicyclic carbocycle in which one ring is benzene and the other is unsaturated is an unsaturated ring system. Saturated carbocyclic rings are also referred to as cycloalkyl rings, e.g., cyclopropyl, cyclobutyl, etc. Unless otherwise noted, carbocycle is unsubstituted or substituted with C₁₋₆ alkyl, C₁₋₆ alkenyl, C₁₋₆ alkynyl, aryl, halogen, NH₂ or OH. A subset of the fused bicyclic unsaturated carbocycles are those bicyclic carbocycles in which one ring is a benzene ring and the other ring is saturated or unsaturated, with attachment via any carbon atom that results in a stable compound. Representative examples of this subset include the following:

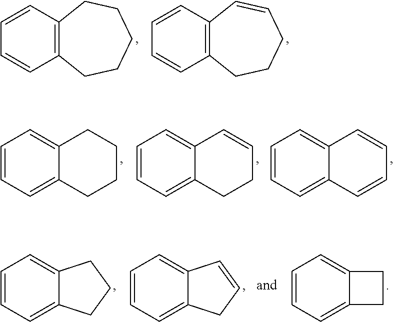

The term “heterocycle” (and variations thereof such as “heterocyclic” or “heterocyclyl”) broadly refers to (i) a stable 4- to 8-membered, saturated or unsaturated monocyclic ring, or (ii) a stable 7- to 12-membered bicyclic ring system, wherein each ring in (ii) is independent of, or fused to, the other ring or rings and each ring is saturated or unsaturated, and the monocyclic ring or bicyclic ring system contains one or more heteroatoms (e.g., from 1 to 6 heteroatoms, or from 1 to 4 heteroatoms) selected from N, O and S and a balance of carbon atoms (the monocyclic ring typically contains at least one carbon atom and the ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Unless otherwise specified, when the heterocyclic ring has substituents, it is understood that the substituents may be attached to any atom in the ring, whether a heteroatom or a carbon atom, provided that a stable chemical structure results.

Non limiting examples of heterocyclic moieties include, but are not limited to, the following: pyrazolyl, azepanyl, azabenzimidazole, benzoimidazolyl, benzofuryl, benzofurazanyl, benzopyrazolyl, benzothiazolyl, benzothienyl, benzotriazolyl, benzothiophenyl, benzoxazolyl, carbazolyl, carbolinyl, chromanyl, cinnolinyl, furanyl, imidazolyl, indolinyl, indolyl, indolazinyl, indazolyl, isobenzofuryl, isochromanyl, isoindolyl, isoquinolyl, isothiazolyl, isoxazolyl, naphthpyridinyl, oxadiazolyl, oxazolyl, oxazolinyl, isooxazolinyl, oxetanyl, pyranyl, pyrazinyl, pyrazolidinyl, pyrazolyl, pyridazinyl, pyridopyridinyl, pyridazinyl, pyridinyl, pyrimidyl, pyrrolyl, quinazolinyl, quinolyl, quinoxalinyl, quinoxalinyl, tetrahydropyranyl, tetrahydroisoquinolinyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuryl, dihydrobenzothiophenyl, dihydrobenzoxazolyl, dihydrofuryl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, tetrahydroquinolinyl, tetrahydroisoquinolinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuryl, tetrahydrothienyl, tetrahydroquinolinyl, 2,3-dihydrobenzofuryl, 2,3-dihydrobenzo-1,4-dioxinyl, imidazo(2,1-b)(1,3)thiazole, and benzo-1,3-dioxolyl.

Heteroaromatics form another subset of the heterocycles; i.e., the term “heteroaromatic” (alternatively “heteroaryl”) generally refers to a heterocycle as defined above in which the entire ring system (whether mono- or poly-cyclic) is an aromatic ring system. The term “heteroaromatic ring” refers a 5- or 6-membered monocyclic aromatic ring or a 7- to 12-membered bicyclic which consists of carbon atoms and one or more heteroatoms selected from N, O and S. In the case of substituted heteroaryl rings containing at least one nitrogen atom (e.g., pyridine), such substitutions can be those resulting in N-oxide formation. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl.

“Hydroxyalkyl” refers to an alkyl group as described above in which one or more (in particular 1 to 3) hydrogen atoms have been replaced by hydroxy groups. Examples include CH₂OH, CH₂CHOH and CHOHCH₃.

“Alkyldiyl,” “alkenyldiyl,” “alkynyldiyl”, “cycloalkyldiyl”, “aryldiyl”, “heteroaryldiyl” and “heterocycloalkyldiyl” refer to a divalent radical obtained by the removal of one hydrogen atom from an alkyl, alkenyl, alkynyl, cycloalkyl, aryl, heteroaryl, and heterocycloalkyl group, respectively, each of which is as defined above.

Unless expressly stated to the contrary, all ranges cited herein are inclusive. For example, a heterocycle described as containing from “1 to 4 heteroatoms” means the heterocycle can contain 1, 2, 3 or 4 heteroatoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds.

The term “substituted” (e.g., as in “aryl which is optionally substituted with one or more substituents . . . ”) includes mono- and poly-substitution by a named substituent to the extent such single and multiple substitution (including multiple substitution at the same site) is chemically allowed.

When any variable (e.g., Rb, etc.) occurs more than one time in any substituent or in Formulas I-II, its definition in each occurrence is independent of its definition at every other occurrence. Also, combinations of substituents and/or variables are permissible only if such combinations result in stable compounds.

In choosing compounds of the present invention, one of ordinary skill in the art will recognize that the various substituents, i.e. R¹, R², R³, etc., are to be chosen in conformity with well-known principles of chemical structure connectivity.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups can be on the same carbon or on different carbons, so long as a stable structure results. The phrase “optionally substituted with one or more substituents” should be taken to be equivalent to the phrase “optionally substituted with at least one substituent” and in such cases one embodiment will have from zero to three substituents.

### Optical Isomers-Diastereomers-Geometric Isomers-Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formulas I and II are shown without a definitive stereochemistry at certain positions. The present invention includes all stereoisomers of Formulas I and II and pharmaceutically acceptable salts and solvates thereof. Unless specifically mentioned otherwise, reference to one isomer applies to any of the possible isomers. Whenever the isomeric composition is unspecified, all possible isomers are included. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I and II may be obtained by stereospecific synthesis using optically pure starting materials or reagents of known configuration.

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH₂C(O)— groups (keto forms) may undergo tautomerism to form hydroxy —CH═C(OH)— groups (enol forms). Both keto and enol forms, individually as well as mixtures thereof, are included within the scope of the present invention.

### Salts

Pharmaceutically acceptable salts include both the metallic (inorganic) salts and organic salts; a list of which is given in Remington's Pharmaceutical Sciences, 17th Edition, pg. 1418 (1985). It is well known to one skilled in the art that an appropriate salt form is chosen based on physical and chemical stability, flowability, hydro-scopicity and solubility. The term “pharmaceutically acceptable salts” refers to salts prepared from pharmaceutically acceptable non-toxic bases or acids. When the compound of the present invention is acidic, its corresponding salt can be conveniently prepared from inorganic bases or organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from organic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, dicyclohexylamine, lysine, methyl-glucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from inorganic or organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methane-sulfonic, mucic, nitric, pamoic, pantothenic, phosphoric, succinic, sulfuric, tartaric, p-toluene-sulfonic acid and the like. Preferred are citric, hydrobromic, hydrochloric, maleic, phosphoric, sulfuric, and tartaric acids.

### Solvates

The present invention includes within its scope solvates of compounds of Formula I and II. As used herein, the term “solvate” refers to a complex of variable stoichiometry formed by a solute (i.e., a compound of Formula I or II) or a pharmaceutically acceptable salt thereof and a solvent that does not interfere with the biological activity of the solute. Examples of solvents include, but are not limited to water, ethanol, and acetic acid. When the solvent is water, the solvate is known as hydrate; hydrate includes, but is not limited to, hemi-, mono, sesqui-, di- and trihydrates.

### Prodrugs

The present invention includes within its scope the use of prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with a compound of Formula I or II, or with a compound which may not be a compound of Formula I or II, but which converts to a compound of Formula I or II in vivo after administration to the patient. Conventional procedures for the selection and preparation of suitable prod rug derivatives are described, for example, in “Design of Prodrugs,” ed. H. Bundgaard, Elsevier, 1985.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I or II. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I or II can be prepared without undue experimentation by conventional techniques well known to those skilled in the art or by processes analogous to those described in the Schemes and Examples herein using appropriate isotopically-enriched reagents and/or intermediates.

### Utilities

Compounds of the present invention are inhibitors of hypoxia-inducible factor (HIF) prolyl hydroxylases, and as such are useful in the treatment and prevention of diseases and conditions in which HIF modulation is desirable, such as anemia and ischemia. Compounds of the invention can be used in a selective and controlled manner to induce hypoxia-inducible factor stabilization and to rapidly and reversibly stimulate erythropoietin production and secretion. Accordingly, another aspect of the present invention provides a method of treating or preventing a disease or condition in a mammal, the treatment or prevention of which is effected or facilitated by HIF prolyl hydroxylase inhibition, which comprises administering an amount of a compound of Formula I or II that is effective for inhibiting HIF prolyl hydroxylase. This aspect of the present invention further includes the use of a compound of Formula I or II in the manufacture of a medicament for the treatment or prevention of a disease or condition modulated by HIF prolyl hydroxylase.

In one embodiment is a method of enhancing endogenous production of erythropoietin in a mammal which comprises administering to said mammal an amount of a compound of Formula I or II that is effective for enhancing endogenous production of erythropoietin.

Another embodiment is a method of treating anemia in a mammal which comprises administering to said mammal a therapeutically effective amount of a compound of Formulas I or II. “Anemia” includes, but is not limited to, chronic kidney disease anemia, chemotherapy-induced anemia (e.g., anemia resulting from antiviral drug regimens for infectious diseases, such as HIV and hepatitis C virus), anemia of chronic disease, anemia associated with cancer conditions, anemia resulting from radiation treatment for cancer, anemias of chronic immune disorders such as rheumatoid arthritis, inflammatory bowel disease, and lupus, and anemias due to menstruation or of senescence or in other individuals with iron processing deficiencies such as those who are iron-replete but unable to utilize iron properly.

Another embodiment is a method of treating ischemic diseases in a mammal, which comprises administering to said mammal a therapeutically effective amount of a compound of Formulas I or II.

### Combination Therapy

Compounds of Formulas I and II may be used in combination with other drugs that are used in the treatment/prevention/suppression or amelioration of the diseases or conditions for which compounds of Formulas I or II are useful. Such other drugs may be administered, by a route and in an amount commonly used therefor, contemporaneously or sequentially with a compound of Formulas I or II. When a compound of Formulas I or II is used contemporaneously with one or more other drugs, a pharmaceutical composition containing such other drugs in addition to the compound of Formulas Igor II is preferred. Accordingly, the pharmaceutical compositions of the present invention include those that also contain one or more other active ingredients, in addition to a compound of Formulas I or II.

### Route of Administration/Dosage

The compounds of this invention can be administered for the treatment or prevention of afflictions, diseases and illnesses according to the invention by any means that effects contact of the active ingredient compound with the site of action in the body of a warm-blooded animal. For example, administration can be oral, topical, including transdermal, ocular, buccal, intranasal, inhalation, intravaginal, rectal, intracisternal and parenteral. The term “parenteral” as used herein refers to modes of administration which include subcutaneous, intravenous, intramuscular, intraarticular injection or infusion, intrasternal and intraperitoneal. For the purpose of this disclosure, a warm-blooded animal is a member of the animal kingdom possessed of a homeostatic mechanism and includes mammals and birds.

The compounds can be administered by any conventional means available for use in conjunction with pharmaceuticals, either as individual therapeutic agents or in a combination of therapeutic agents. They can be administered alone, but are generally administered with a pharmaceutical carrier selected on the basis of the chosen route of administration and standard pharmaceutical practice.

The dosage administered will be dependent on the age, health and weight of the recipient, the extent of disease, kind of concurrent treatment, if any, frequency of treatment and the nature of the effect desired. Usually, a daily dosage of active ingredient compound will be from about 0.1-2000 milligrams per day. Ordinarily, from 10 to 500 milligrams per day in one or more applications is effective to obtain desired results. These dosages are the effective amounts for the treatment and prevention of afflictions, diseases and illnesses described above, e.g., anemia.

### Pharmaceutical Composition

Another aspect of the present invention provides pharmaceutical compositions which comprises a compound of Formulas I or II and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formulas I or II, additional active ingredient(s), and pharmaceutically acceptable excipients.

The pharmaceutical compositions of the present invention comprise a compound represented by Formulas I or II (or a pharmaceutically acceptable salt or solvate thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The active ingredient can be administered orally in solid dosage forms, such as capsules, tablets, troches, dragées, granules and powders, or in liquid dosage forms, such as elixirs, syrups, emulsions, dispersions, and suspensions. The active ingredient can also be administered parenterally, in sterile liquid dosage forms, such as dispersions, suspensions or solutions. Other dosages forms that can also be used to administer the active ingredient as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene gycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorobutanol.

Suitable pharmaceutical carriers are described in Remington's Pharmaceutical Sciences, A. Osol, a standard reference text in this field.

For administration by inhalation, the compounds of the present invention may be conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of a compound of Formulas I or II in suitable propellants, such as fluorocarbons or hydrocarbons.

For ocular administration, an ophthalmic preparation may be formulated with an appropriate weight percent solution or suspension of the compounds of Formulas I or II in an appropriate ophthalmic vehicle, such that the compound is maintained in contact with the ocular surface for a sufficient time period to allow the compound to penetrate the corneal and internal regions of the eye.

Useful pharmaceutical dosage-forms for administration of the compounds of this invention include, but are not limited to, hard and soft gelatin capsules, tablets, parenteral injectables, and oral suspensions.

A large number of unit capsules are prepared by filling standard two-piece hard gelatin capsules each with 100 milligrams of powdered active ingredient, 150 milligrams of lactose, 50 milligrams of cellulose, and 6 milligrams magnesium stearate.

A mixture of active ingredient in a digestible oil such as soybean oil, cottonseed oil or olive oil is prepared and injected by means of a positive displacement pump into gelatin to form soft gelatin capsules containing 100 milligrams of the active ingredient. The capsules are washed and dried.

A large number of tablets are prepared by conventional procedures so that the dosage unit is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

A parenteral composition suitable for administration by injection is prepared by stirring 1.5% by weight of active ingredient in 10% by volume propylene glycol. The solution is made to volume with water for injection and sterilized.

An aqueous suspension is prepared for oral administration so that each 5 milliliters contain 100 milligrams of finely divided active ingredient, 100 milligrams of sodium carboxymethyl cellulose, 5 milligrams of sodium benzoate, 1.0 grams of sorbitol solution, U.S.P., and 0.025 milliliters of vanillin.

The same dosage forms can generally be used when the compounds of this invention are administered stepwise or in conjunction with another therapeutic agent. When drugs are administered in physical combination, the dosage form and administration route should be selected depending on the compatibility of the combined drugs. Thus the term coadministration is understood to include the administration of the two agents concomitantly or sequentially, or alternatively as a fixed dose combination of the two active components.

Compounds of the invention can be administered as the sole active ingredient or in combination with a second active ingredient, including other active ingredients known to be useful for improving the level of erythropoietin in a patient.

### Abbreviations Used in the Description of the Preparation of the Compounds

˜ Approximately
Aq Aqueous
BnOH Benzylalcohol
Boc₂O or di-tert-butyl dicarbonate BOC₂O
Brine Saturated aqueous sodium chloride solution
DCM Dichloromethane
DEA Diethylamine
DIPEA N,N-diisopropylethylaime
DMA Dimethylacetamide
DMF N,N-dimethylformamide
DMSO Dimethyl sulfoxide
EDC or EDCI 1-(3-dimethylaminopropyl)-3-ethylcarbodiimide hydrogenchloride salt
EtOAc or EA Ethyl acetate
Et (et) Ethyl
EtOH Ethanol
Et₂O or ether Diethyl ether
Et₃N triethylamine
g Gram
h or hr Hour
HATU O-(7-Azabenzotriazol-1-yl)-N,N,N′,N′-tetramethyluronium hexafluorophosphate
HCl Hydrochloric acid
HOAt 3-hydroxytriazolo[4,5,b]pyridine
HPLC High-performance liquid chromatography
i-propanol Isopropyl alcohol
i-PrOH or IPA Isopropyl alcohol
K₃PO₄ Potassium phosphate
LCMS Liquid chromatography mass spectrometry
LiOH Lithium hydroxide
Mg Milligrams
mL Milliliters
mmol Millimole
MeOH Methanol
min Minutes
ms or MS Mass spectrum
μg Microgram(s)
μL Microliters
NaH Sodium hydride
NaHCO₃ Sodium bicarbonate
NaOAc Sodium acetate
Na₂SO₄ Sodium sulfate
NaOH Sodium hydroxide
NH₄OH ammonium hydroxide
Pd/C Palladium on carbon
PdCl₂(dppf) [1,1′-Bis(diphenylphosphino)ferrocene]dichloropalladium(II)
PCl₅ Phosphoril chloride
Ph Phenyl group
Rₜ Retention time
RT or rt Room temperature
SFC Supercritical fluid chromatography
^{t}Bu Tert-butyl
TEA Triethylamine
TFA Trifluoroacetic acid
THF Tetrahydrofuran
TLC Thin layer chromatography

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound in place of multiple substituents which are allowed under the definitions of Formula I or II defined previously.

### General Experimental Comments

Reactions sensitive to moisture or air were performed under nitrogen using anhydrous solvents and reagents. The progress of reactions was determined by either analytical thin layer chromatography (TLC) performed with E. Merck® precoated TLC plates, silica gel 60F-254, layer thickness 0.25 mm or liquid chromatography-mass spectrum (LC-MS). Mass analysis was performed on a Waters Micromass® ZQ™ (Waters Corporation, Milford, Mass.) with electrospray ionization in positive ion detection mode. High performance liquid chromatography (HPLC) was conducted on an Agilent 1100™ series HPLC (Agilent Technologies, Santa Clara, Calif.) on Waters C18 XTerra™ (Waters Corporation, Milford, Mass.) 3.5 m 3.0×50 mm column with gradient 10:90-100 v/v CH₃CN/H₂O+v 0.05% TFA over 3.75 min then hold at 100 CH₃CN+v 0.05% TFA for 1.75 min; flow rate 1.0 mL/min, UV wavelength 254 nm). Concentration of solutions was carried out on a rotary evaporator under reduced pressure. Flash chromatography was performed using a Biotage® Flash Chromatography apparatus (Dyax Corp., Charlottesville, Va.) on silica gel (32-63 mM, 60 Å pore size) in pre-packed cartridges. ¹H-NMR spectra were obtained on a 400 or 500 MHz VARIAN® Spectrometer (Agilent Technologies, Santa Clara, Calif.) in CDCl₃ or CD₃OD or other solvents as indicated and chemical shifts are reported as 8 using the solvent peak as reference and coupling constants are reported in hertz (Hz).

### Biological Assays

The exemplified compounds of the present invention have been found to inhibit the hydroxylation of a HIF peptide by PHD2 and exhibit IC₅₀ values ranging between 0.1 nanomolar to 10 micromolar. Select examples of assays that may be used to detect favorable activity are disclosed in the following publications: Oehme, F., et al., Anal. Biochem. 330:74-80 (2004); Hirsilä, M, et al., J. Bio. Chem. 278 (33): 30772-30780 (2005); Hyunju, C., et al., Biochem. Biophys. Res. Comm. 330 275-280 (2005); and Hewitson, K. S., et al., Methods in Enzymology, (Oxygen Biology and Hypoxia); Elsevier Publisher (2007), pg. 25-42 (ISSN: 0076-6879).

The biological activity of the present compounds may be evaluated using assays described herein below:

To each well of a 384-well plate, 1 μL of test compounds in DMSO (final concentration ranging from 0.3 nM to 10 μM) were added into 20 μl of assay buffer (50 mM Tris pH 7.4/0.01% Tween-20/0.1 mg/ml bovine serum albumin/10 μM ferrous sulfate/1 mM sodium ascorbate/20 μg/ml catalase) containing 0.15 μg/ml FLAG-tagged full length PHD2 expressed in and purified from baculovirus-infected Sf9 cells. After a 5 min preincubation at room temperature, the enzymatic reactions were initiated by the addition of 4 μL of substrates {final concentrations of 0.2 μM 2-oxoglutarate and 0.5 μM HIF-1α peptide biotinyl-DLDLEMLAPYIPMDDDFQL (SEQ ID NO: 1)}. After incubation for 45 minutes at room temperature, the reactions were terminated by the addition of a 25 μL quench/detection mix to a final concentration of 1 mM ortho-phenanthroline, 0.1 mM EDTA, 0.5 nM anti-(His)6 LANCE reagent (Perkin-Elmer Life Sciences), 100 nM AF647-labeled streptavidin (Invitrogen), and 2 μg/ml (His)6-VHL complex {S. Tan Protein Expr. Purif. 21, 224-234 (2001)} and the signals were developed for 30 minutes at room temperature. The ratio of time resolved fluorescence signals at 665 and 620 nm was determined, and percent inhibition was calculated relative to the high control samples (DMSO treated) run in parallel, after background subtraction.

Inhibition of the catalytic activity of HIF-PHD1 and HIF-PHD3 can be determined similarly, except for HIF-PHD3, final concentrations of 4 μM 2-oxoglutarate is used during the reaction.

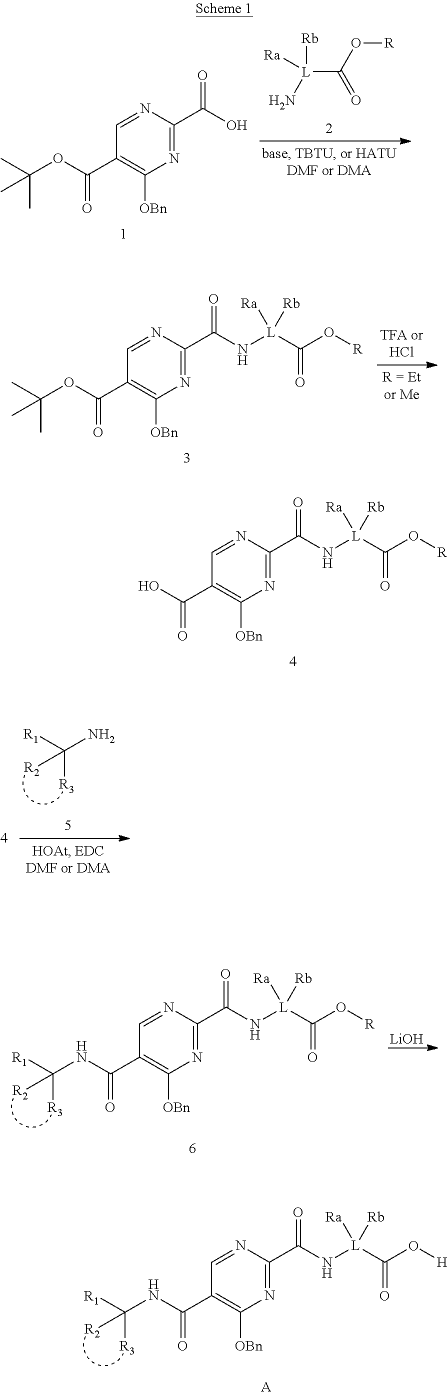

Scheme 1 outlines the general synthetic sequence for compounds of Formula A. The condensation of 1 with amino acid 2 gives compound 3. Hydrolysis of butyl ester and the removal of benzyl group of 3 in acidic condition produces compound 4. Amide formation between acid 4 and amine 5 provides 6 which is hydrolyzed to afford compounds of Formula A.

Alternatively, compounds of Formula A can be prepared according to Scheme 2 where the removal of benzyl group is realized via palladium catalyzed hydrogenation.

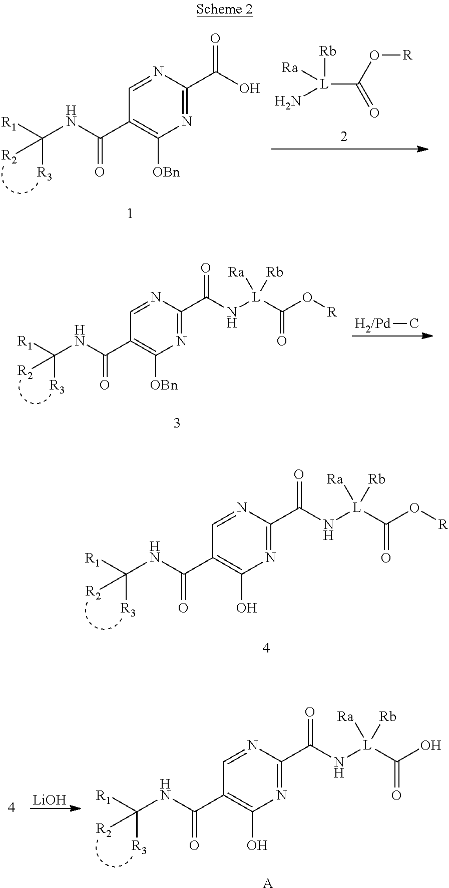

Starting materials useful for the preparation of the compounds of the present invention are either commercially available, known in the literature (reference provided) or may be prepared using chemical methodologies known to those skilled in the art.

### Intermediate 1

### 2-(4′-(Trifluoromethyl)-[1,1′-biphenyl]-4-yl)propan-2-amine (I-1)

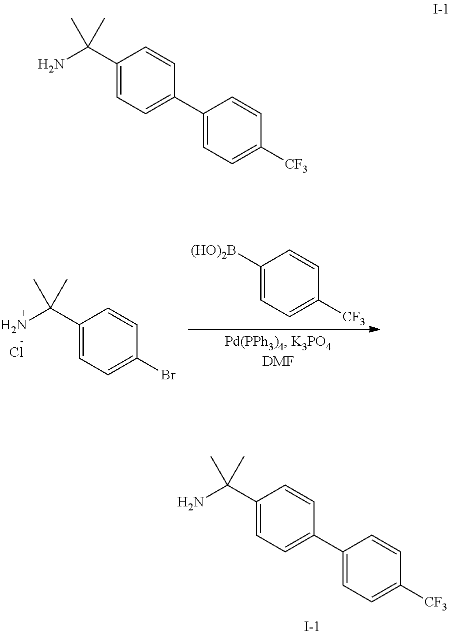 [SMILES: CC(C)(N)c1ccc(-c2ccc(C(F)(F)F)cc2)cc1 ; CC(C)(N)c1ccc(Br)cc1 ; Cl ; OB(O)c1ccc(C(F)(F)F)cc1 ; CC(C)(N)c1ccc(-c2ccc(C(F)(F)F)cc2)cc1 ; [I-]]

To a solution of 2-(4-bromophenyl)propan-2-aminium chloride (100 mg, 0.32 mmol) in DMF (2.0 mL) was added (4-(trifluoromethyl)phenyl)boronic acid (120 mg, 0.64 mmol) and aq. potassium carbonate (2M, 0.32 mL, 0.64 mmol). After replacing the air inside the container with nitrogen, to the mixture was added Pd(Ph₃P)₄ (37 mg, 0.03 mmol). The mixture was stirred at 90° C. for 16 h. When LCMS showed the reaction completed, the mixture was filtered and the filtrate was concentrated under vacuum to remove DMF. The residue was purified by prep. TLC (eluted with EtOAc:MeOH:NH₄OH=10:1:0.01) to afford 2-(4′-(trifluoromethyl)-[1,1′-biphenyl]-4-yl)propan-2-amine. ¹HNMR (CDCl₃, 400 MHz) δ: 7.66-7.64 (m, 6H), 7.57 (d, J=8.4 Hz, 2H), 1.72 (s, 6H).

Intermediates I-2 through I-9 in Table 1 were prepared using procedures analogous to the procedure described for Intermediate I-1 with the substitution of appropriate starting materials.

TABLE 1

| Intermediate | | | MS m/z |
|---|---|---|---|
| Number | Name | Structure | (M + 1)⁺ |
| I-2 | 2-(4-(isoquinolin-5- yl)phenyl)propan-2-amine | 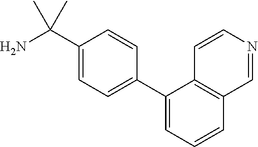 [SMILES: CC(C)(N)c1ccc(-c2cccc3cnccc23)cc1] | (M + 1)⁺ 263 |
| I-3 | 2-(4-(pyridin-3- yl)phenyl)propan-2-amine | 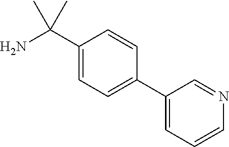 [SMILES: CC(C)(N)c1ccc(-c2cccnc2)cc1] | (M + 1)⁺ 213 |
| I-4 | 4′-(2-aminopropan-2-yl)-2- methyl-[1,1′-biphenyl]-4- carbonitrile | 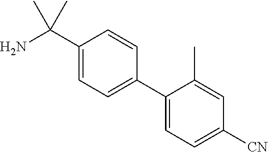 | (M + 1)⁺ 263 |
| I-5 | 2-(4′-fluoro-[1,1′-biphenyl-4- yl)propan-2-amine | 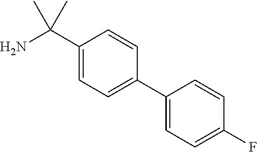 [SMILES: CC(C)(N)c1ccc(-c2ccc(F)cc2)cc1] | (M + 1)⁺ 230 |
| I-6 | 2-(4′-(trifluoromethoxy)-[1,1′- biphenyl]-4-yl)propan-2- amine | 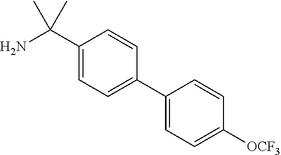 [SMILES: CC(C)(N)c1ccc(-c2ccc(OC(F)(F)F)cc2)cc1] | (M + 1)⁺ 296 |
| I-7 | 2-(4-(6-methoxypyridin-3- yl)phenyl)propan-2-amine | 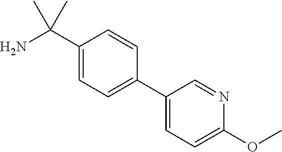 [SMILES: COc1ccc(-c2ccc(C(C)(C)N)cc2)cn1] | (M + 1)⁺ 243 |
| I-8 | 2-(4-(1H-pyrazol-3- yl)phenyl)propan-2-amine | 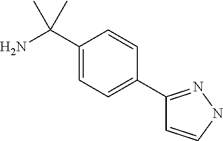 | (M + 1)⁺ 202 |
| I-9 | 2-(4′-chloro-[1,1′-biphenyl]-4- yl)propan-2-amine | 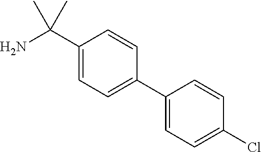 [SMILES: CC(C)(N)c1ccc(-c2ccc(Cl)cc2)cc1] | (M + 1)⁺ 246 |

### Intermediate 10

### 1-(4′-(Trifluoromethyl)-[1,1′-biphenyl]-4-yl)cyclopropanamine (I-10)

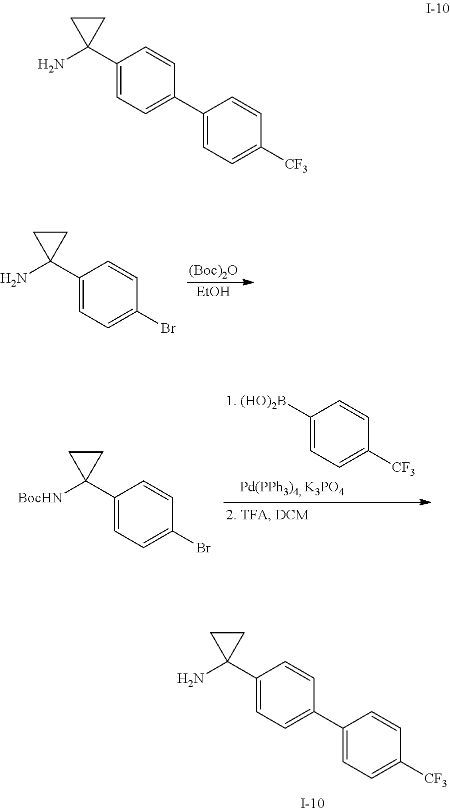

### Step A tert-butyl (1-(4-bromophenyl)cyclopropyl)carbamate

To a solution of 1-(4-bromophenyl)cyclopropanamine (2 g, 9.4 mmol) in ethanol (20 mL) was added (BOC)₂O (4.4 mL, 19 mmol), then the mixture was stirred at room temperature for 16 h. When TLC showed that the reaction was complete, the mixture was concentrated under reduced pressure. The residue was triturated with petroleum ether (20 mL), and the solid was collected by suction to afford crude product tert-butyl (1-(4-bromophenyl)cyclopropyl)carbamate. ¹HNMR (CDCl₃, 400 MHz) δ: 7.38 (d, J=8.0 Hz, 2H), 7.08 (d, J=8.0 Hz, 2H), 5.23 (br s, 1H), 1.42 (s, 9H), 1.26-1.17 (m, 4H).

### Step B tert-butyl (1-(4′-(trifluoromethyl)-[1,1′-biphenyl]-4-yl)cyclopropyl)carbamate

To a solution of tert-butyl (1-(4-bromophenyl)cyclopropyl)carbamate (50 mg, 0.16 mmol) in DMF (2 mL) was added (4-(trifluoromethyl)phenyl)boronic acid (60 mg, 0.32 mmol) and aq. potassium carbonate (2M, 0.32 mL, 0.64 mmol). After degassing with nitrogen, Pd(Ph₃P)₄ (37 mg, 0.03 mmol) was added to the mixture. Then the mixture was stirred at 90° C. for 16 h. When LCMS showed that the reaction was completed, the mixture was filtered and the filtrate was concentrated in vacuum to remove DMF. The residue was purified by prep. TLC (eluted with petroleum ether:EtOAc=5:1) to afford tert-butyl (1-(4′-(trifluoromethyl)-[1,1′-biphenyl]-4-yl)cyclopropyl)carbamate. ¹H NMR (CDCl₃, 400 MHz) δ: 7.66-7.64 (m, 4H), 7.52 (d, J=8.0 Hz, 2H), 7.29 (d, J=8.4 Hz, 2H), 5.29 (br s, 1H), 1.45 (s, 9H), 1.32-1.28 (m, 4H).

To a solution of tert-butyl(1-(4′-(trifluoromethyl)-[1,1′-biphenyl]-4-yl)cyclopropyl)carbamate (20 mg, 0.05 mmol) in DCM (2 mL) was added TFA (0.4 mL, 0.53 mmol). Then the mixture was stirred at room temperature for 3 h. When TLC showed that the reaction was complete, the mixture was concentrated in vacuum to afford the crude product 1-(4′-(trifluoromethyl)-[1,1′-biphenyl]-4-yl)cyclopropanamine, 1-10.

Intermediates I-11 through I-14 disclosed in Table 2 were prepared under analogous procedures as describe for the synthesis of Intermediate I-10 but by substituting appropriate starting materials.

TABLE 2

| Intermediate | | | MS m/z |
|---|---|---|---|
| Number | Compound Name | Structure | (M + 1)⁺ |
| I-11 | 1-(4′-chloro-[1,1′-biphenyl]-4- yl)cyclopropanamine | 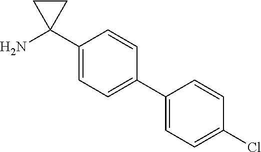 [SMILES: NC1(c2ccc(-c3ccc(Cl)cc3)cc2)CC1] | (M + 1)⁺ 244 |
| I-12 | 1-(4-(isoquinolin-5- yl)phenyl)cyclopropanamine | 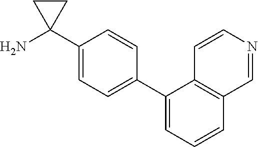 [SMILES: NC1(c2ccc(-c3cccc4cnccc34)cc2)CC1] | (M + 1)⁺ 261 |
| I-13 | 1-(4′-fluoro-[1,1′-biphenyl]-4- yl)cyclopropanamine | 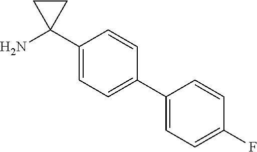 [SMILES: NC1(c2ccc(-c3ccc(F)cc3)cc2)CC1] | (M + 1)⁺ 228 |
| I-14 | 1-(4-(6-methoxypyridin-3- yl)phenyl)cyclopropanamine | 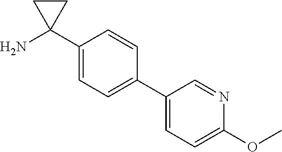 [SMILES: COc1ccc(-c2ccc(C3(N)CC3)cc2)cn1] | (M + 1)⁺ 241 |

### Example 1 and Example 2

### (R)-3-(5-(Benzhydrylcarbamoyl)-4-hydroxypyrimidine-2-carboxamido)-2-methyl propanoic acid and (S)-3-(5-(Benzhydrylcarbamoyl)-4-hydroxypyrimidine-2-carboxamido)-2-methyl propanoic acid

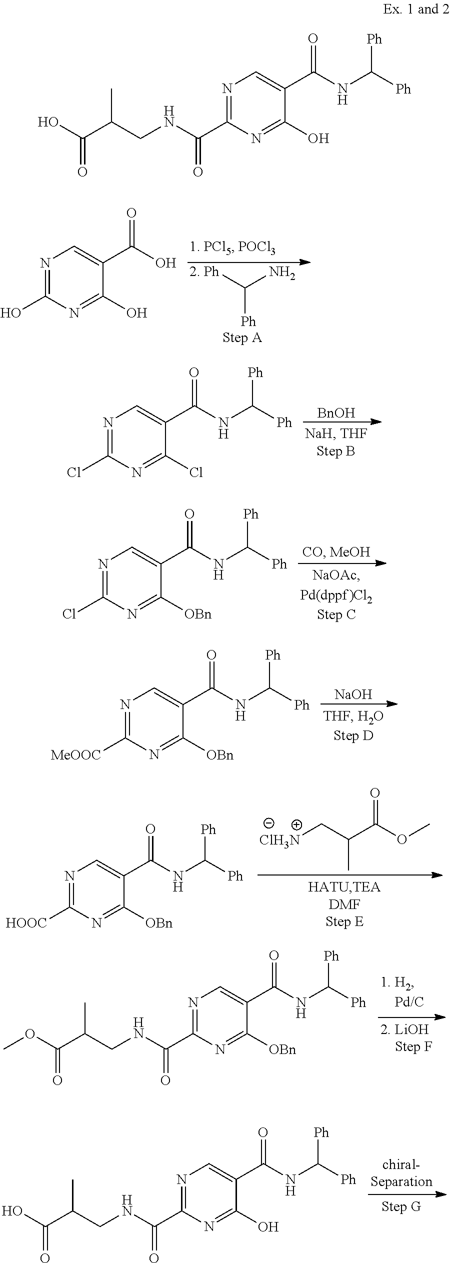

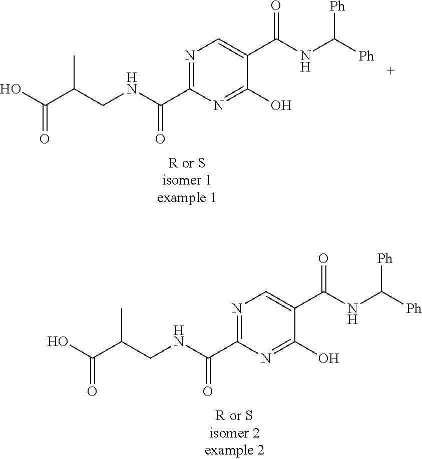 [SMILES: CC(CNC(=O)c1ncc(C(=O)NC(c2ccccc2)c2ccccc2)c(O)n1)C(=O)O ; CC(CNC(=O)c1ncc(C(=O)NC(c2ccccc2)c2ccccc2)c(O)n1)C(=O)O]

### Step A N-Benzhydryl-2,4-dichloropyrimidine-5-carboxamide

To a 1 L flask was added POCl₃ (100 mL), followed by 2,4-dihydroxypyrimidine-5-carboxylic acid (10 g, 0.064 mol) and PCl₅ (14.7 g, 0.071 mol). The mixture was refluxed for 6 hours. After concentration, the residue was co-evaporated with toluene (100 mL) twice to remove residue POCl₃. The residue was then dissolved in DCM (100 mL). The resulting solution was added dropwise to a solution of diphenylmethanamine (12.9 g, 0.07 mol) and TEA (21 g, 0.2 mol) in anhydrous DCM (400 mL) at ˜0° C. After stirring for 30 min at rt, the mixture was washed with water (200 mL) and the precipitate was collected via suction. The filter cake was then dissolved in EtOAc (400 mL) and the solution was washed with hydrochloric acid (5%, 200 mL), water (200 mL) and brine (100 mL), dried over anhydrous Na₂SO₄ and then concentrated to afford the intermediate, N-Benzhydryl-2,4-dichloropyrimidine-5-carboxamide. ¹H NMR (CDCl₃, 400 MHz) δ 8.98 (s, 1H), 7.39-7.29 (m, 10H), 7.23 (d, J=7.3 Hz, 1H), 6.41 (d, J=7.7 Hz, 1H). LC/MS (m/z): 358 (M+H)⁺.

### Step B N-Benzhydryl-4-(benzyloxy)-2-chloropyrimidine-5-carboxamide

To a solution of benzyl alcohol (2.5 ml, 23.9 mmol) in anhydrous THF (100 ml) was added NaH (1.0 g, 26.0 mmol) slowly at 0° C., then the reaction mixture was allowed to stir at room temperature for 0.5 h. The resulting suspension was then added slowly to an ice-salt-cooled solution of N-benzhydryl-2,4-dichloropyrimidine-5-carboxamide (10.0 g, 21.7 mmol) in THF (150 mL) through an addition funnel, so as to keep the reaction temperature at or below 0° C. After addition was complete, the reaction mixture was stirred at 0° C. for 1 hour, when LCMS showed the reaction completed. The reaction mixture was acidified using 5% HCl at ˜0° C. to pH 6-7, and the mixture was extracted with EtOAc (200 mL). The organic layer was washed with water and brine (200 mL each), dried over Na₂SO₄ and concentrated under vacuum to afford crude product, which was purified through recrystallization from EtOAc/petroleum ether to afford N-benzhydryl-4-(benzyloxy)-2-chloropyrimidine-5-carboxamide. ¹H NMR (CDCl₃ 400 MHz) δ 9.17 (s, 1H), 8.11 (d, J=7.9 Hz, 1H), 7.39-7.33 (m, 5H), 7.22-7.19 (m, 6H), 7.05-7.03 (m, 4H), 6.34 (d, J=8.2 Hz, 1H), 5.53 (s, 2H). LC/MS (m/z): 429 (M+H)⁺.

### Step C Methyl 5-(benzhydrylcarbamoyl)-4-(benzyloxy)pyrimidine-2-carboxylate

To a 2 L stainless steel autoclave was added N-benzhydryl-4-(benzyloxy)-2-chloropyrimidine-5-carboxamide (20.0 g, 46.6 mmol), Pd(dppf)Cl₂ (3.4 g, 4.66 mmol), NaOAc (11.6 g, 139.8 mmol) and MeOH (800 mL). The air in the autoclave was replaced with carbon monoxide and the pressure was adjusted to 3.6 MPa. Then the reaction mixture was stirred at 70° C. for 3 hours. After cooling, the filtrate was concentrated under vacuum. The residue was purified by column chromatography on silica gel (eluted with petroleum ether/EtOAc 3:1 to 1:1) to afford methyl 5-(benzhydrylcarbamoyl)-4-(benzyloxy)pyrimidine-2-carboxylate. ¹H NMR (CDCl₃, 400 MHz) δ 9.42 (s, 1H), 8.29 (d, J=7.9 Hz, 1H), 7.40-7.33 (m, 5H), 7.23-7.21 (m, 6H), 7.07-7.04 (m, 4H), 6.35 (d, J=7.9 Hz, 1H), 5.64 (s, 2H), 4.05 (s, 3H). LC/MS (m/z): 454 (M+H)⁺.

### Step D 5-(Benzhydrylcarbamoyl)-4-(benzyloxy)pyrimidine-2-carboxylic acid

To a solution of methyl 5-(benzhydrylcarbamoyl)-4-(benzyloxy)pyrimidine-2-carboxylate (10.0 g, 22.8 mmol) in THF (350 mL) was added aq. NaOH (5%, 22 mL, 27 mmol) over a 50 min period. After addition, the reaction mixture was allowed to stir at rt for 30 min, when TLC showed that the reaction was complete. The mixture was acidified with aq. HCl (5%) to pH 3-4 and extracted with DCM (500 mL), washed with brine (300 mL). The organic layer was dried over Na₂SO₄, concentrated under vacuum to about 50 mL, and to the residue was then added petroleum ether (200 mL), the precipitate was collected by filtration to afford 5-(benzhydrylcarbamoyl)-4-(benzyloxy)pyrimidine-2-carboxylic acid. ¹H NMR (CDCl₃, 400 MHz) δ 9.34 (s, 1H), 8.21 (d, J=7.7 Hz, 1H), 7.36-7.29 (m, 5H), 7.19-7.17 (m, 6H), 7.00-6.98 (m, 4H), 6.29 (d, J=8.2 Hz, 1H), 5.66 (s, 2H). LC/MS (m/z): 440 (M+H)⁺.

### Step E Methyl 3-(5-(benzhydrylcarbamoyl)-4-(benzyloxy)pyrimidine-2-carboxamido)-2-methylpropanoate

To a solution of 5-(benzhydrylcarbamoyl)-4-(benzyloxy)pyrimidine-2-carboxylic acid (600 mg, 1.4 mmol) in DMF (10 mL) was added racemic methyl 3-amino-2-methylpropanoate hydrochloride (420 mg, 2.7 mmol), TEA (270 mg, 2.6 mmol) and HATU (610 mg, 1.6 mmol). The mixture was stirred at room temperature overnight. When TLC showed that the reaction was complete, the reaction mixture was washed with water (20 mL), extracted with EtOAc (40 mL). The organic layer was washed with brine (20 mL), dried over sodium sulfate, filtered and concentrated under vacuum. The residue was purified by prep. TLC (DCM/MeOH=15:1) to afford methyl 3-(5-(benzhydrylcarbamoyl)-4-(benzyloxy)pyrimidine-2-carboxamido)-2-methylpropanoate. ¹H NMR (CDCl₃, 400 MHz) δ 9.30 (s, 1H), 8.38 (t, J=6.2 Hz, 1H), 8.23 (d, J=8.0 Hz, 1H), 7.36-7.27 (m, 5H), 7.18-7.13 (m, 6H), 7.02-6.96 (m, 4H), 6.29 (d, J=8.0 Hz, 1H), 5.61 (s, 2H), 4.05 (q, J=7.1 Hz, 1H), 3.65 (s, 3H), 3.53 (ddd, J=6.2, 7.8, 13.8 Hz, 1H), 2.79 (dt, J=4.6, 7.5 Hz, 1H), 1.22-1.19 (m, 3H). LC/MS (m/z): 539 (M+H)⁺.

### Step F 3-(5-(benzhydrylcarbamoyl)-4-hydroxypyrimidine-2-carboxamido)-2-methylpropanoic acid

To a solution of methyl 3-(5-(benzhydrylcarbamoyl)-4-(benzyloxy)pyrimidine-2-carboxamido)-2-methylpropanoate (410 mg, 0.8 mmol) in EtOAc (20 mL) was added Pd/C (Wet, 10%, 50 mg). The mixture was stirred under hydrogen atmosphere for 1 h. When TLC analysis showed that the reaction was complete, the reaction mixture was filtered through a pad of Celite, and the filtrate was concentrated under reduced pressure to afford methyl 3-(5-(benzhydrylcarbamoyl)-4-(benzyloxy)pyrimidine-2-carboxamido)-2-methyl propanoate. LC/MS (m/z): 439 (M+H)⁺. To the above product (100 mg, 0.22 mmol) in THF (3 mL) was added aq. LiOH (0.5 mL, 0.5 mmol). After addition, the reaction mixture was allowed to stir at rt for 30 min, when TLC that showed the reaction was completed. The mixture was acidified with aq. HCl (5%) to pH 3-4. The precipitate was collected by filtration to afford 2-carboxamido)-2-methylpropanoic acid. LC/MS (m/z): 435 (M+H)⁺.

### Step G (R)-3-(5-(benzhydrylcarbamoyl)-4-hydroxypyrimidine-2-carboxamido)-2-methyl and (S)-3-(5-(benzhydrylcarbamoyl)-4-hydroxypyrimidine-2-carboxamido)-2-methyl propanoic acid

The enantiomers of Step F were resolved by SFC (SFC condition: Chiralpak® AD-H 250*4.6 mm I.D., 5 um (Chiral Technologies, Inc. West Chester, Pa.); 40% iPrOH (0.05% DEA) in CO₂; 2.35 mL/min 220 nm) to give:

### Example 1 (Isomer 1, RT 2.610 Min)

¹H NMR (Methanol-d₄, 400 MHz) δ 8.65 (br d, J=8.0 Hz, 1H), 7.24-7.16 (m, 10H), 6.22 (d, J=8.0 Hz, 1H), 3.52-3.47 (m, 1H), 3.42-3.37 (m, 1H), 2.71-2.66 (m, 1H), 1.10 (d, J=7.2 Hz, 3H). LC/MS (m/z): 435 (M+H)⁺. Human HIF-PHD2 IC₅₀: 2.3 nM.

### Example 2 (Isomer 2, RT 2.991 Min)

LC/MS (m/z): 435 (M+H)⁺. Human HI-PHD2 IC₅₀: 1.7 nM.

### Example 3

### 3-(5-(Benzhydrylcarbamoyl)-4-hydroxypyrimidine-2-carboxamido)-2-hydroxy-2-methylpropanoic acid (Ex. 3)

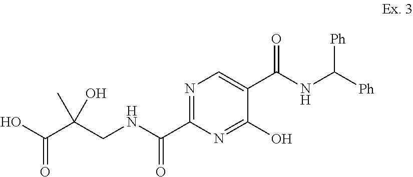 [SMILES: CC(O)(CNC(=O)c1ncc(C(=O)NC(c2ccccc2)c2ccccc2)c(O)n1)C(=O)O]

### Step A Ethyl 3-(5-(benzhydrylcarbamoyl)-4-(benzyloxy)pyrimidine-2-carboxamido)-2-hydroxy-2-methylpropanoate

To a 100 mL single-neck flask equipped with a stirring bar was added ethyl 3-amino-2-hydroxy-2-methylpropanoate hydrochloride (251 mg, 1.3 mmol) and DMF (8 mL). To this solution was added DIPEA (351 mg, 2.7 mmol), 5-(benzhydrylcarbamoyl)-4-(benzyloxy)pyrimidine-2-carboxylic acid (Example 1 Step D, 300 mg, 0.7 mmol) and HATU (517 mg, 1.3 mmol) at room temperature. The mixture was stirred at rt for 16 h, poured into water, and then extracted with EtOAc (200 mL). The organic layers were washed with water and brine, dried over anhydrous sodium sulfate and concentrated under vacuum. The residue was purified by column chromatography on silica gel (eluted with Petroleum Ether/EtOAc 1:1-1:2) to afford ethyl 3-(5-(benzhydrylcarbamoyl)-4-(benzyloxy)pyrimidine-2-carboxamido)-2-hydroxy-2-methylpropanoate. ¹H NMR (CDCl₃, 400 MHz) δ 9.37 (s, 1H), 8.32-8.21 (m, 2H), 7.40-7.05 (m, 15H), 6.35 (d, J=8.0 Hz, 1H), 5.64 (s, 2H), 4.25 (q, J=7.2 Hz, 2H), 4.03-3.98 (m, 1H), 3.62 (br s, 1H), 3.61-3.58 (m, 1H), 1.48 (s, 3H), 1.29 (t, J=7.2 Hz, 3H). LC/MS (m/z): 569 (M+H)⁺.

### Step B Ethyl 3-(5-(benzhydrylcarbamoyl)-4-hydroxypyrimidine-2-carboxamido)-2-hydroxy-2-methylpropanoate

To a solution of ethyl 3-(5-(benzhydrylcarbamoyl)-4-(benzyloxy)pyrimidine-2-carboxamido)-2-hydroxy-2-methylpropanoate (200 mg, 0.35 mmol) in EtOAc (20 mL) was added Pd/C (Wet, 10%, 50 mg). The mixture was stirred under a hydrogen atmosphere for 1 h. When TLC showed that the reaction was complete, the reaction mixture was filtered through a pad of Celite, and the filtrate was concentrated under reduced pressure to afford ethyl 3-(5-(benzhydrylcarbamoyl)-4-hydroxypyrimidine-2-carboxamido)-2-hydroxy-2-methylpropanoate. ¹H NMR (CDCl₃, 400 MHz) δ 11.10 (br s, 1H), 10.38 (br d, J=8.0 Hz, 1H), 8.97 (s, 1H), 8.14 (br, 1H), 7.32-7.22 (m, 10H), 6.45 (d, J=8.0 Hz, 1H), 4.25 (quar, J=6.8 Hz, 2H), 3.94-3.91 (m, 1H), 3.65 (brs, 1H), 3.57-3.52 (m, 1H), 1.46 (s, 3H), 1.29 (t, J=6.8 Hz, 3H). LC/MS (m/z): 479 (M+H)⁺.

### Step C 3-(5-(benzhydrylcarbamoyl)-4-hydroxypyrimidine-2-carboxamido)-2-hydroxy-2-methylpropanoic acid

To a solution of ethyl 3-(5-(benzhydrylcarbamoyl)-4-hydroxypyrimidine-2-carboxamido)-2-hydroxy-2-methylpropanoate (120 mg, 0.25 mmol) in THF (8 mL) was added aq. LiOH (1M, 2 mL, 2 mmol) at RT. The mixture was stirred at rt for 20 min. When LCMS showed that the reaction was completed, the mixture was concentrated to remove THF, and the aqueous residue was acidified with 5% hydrochloric acid to pH=2. The precipitate was collected by filtration to give 3-(5-(benzhydrylcarbamoyl)-4-hydroxypyrimidine-2-carboxamido)-2-hydroxy-2-methylpropanoic acid. ¹H NMR (DMSO-d₆, 400 MHz): δ 10.40 (br, 1H), 8.54-8.51 (m, 2H), 7.34-7.24 (m, 10H), 6.24 (d, J=8.0 Hz, 1H), 5.64 (brs, 1H), 3.61-3.56 (m, 2H), 1.26 (s, 3H). LC/MS (m/z): 451 (M+H)⁺. Human HI-PHD2 IC₅₀: 4.1 nM.

Examples 4 through 7 in Table 3 were prepared under analogous procedures to those described for the synthesis of Example 3, but by substituting appropriate starting materials.

TABLE 3

| | | | MS m/z |
|---|---|---|---|
| | | | (M + 1)⁺ and |
| Example | | | human HIF- |
| Number | Name | Structure | PHD2 IC₅₀ |
| Ex. 4 | 1-((5-(benzhydrylcarbamoyl)- 4-hydroxypyrimidine-2- carboxamido)methyl) cyclopropanecarboxylic acid | 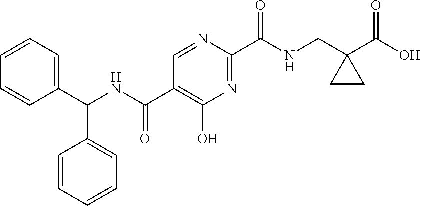 [SMILES: O=C(NCC1(C(=O)O)CC1)c1ncc(C(=O)NC(c2ccccc2)c2ccccc2)c(O)n1] | (M + 1)⁺ 447 IC₅₀ 8.5 nM |
| Ex. 5 | 3-(5-(benzhydrylcarbamoyl)- 4-hydroxypyrimidine-2- carboxamido)-2- hydroxypropanoic acid | 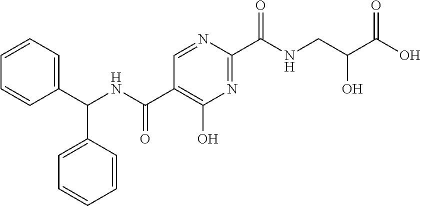 [SMILES: O=C(NCC(O)C(=O)O)c1ncc(C(=O)NC(c2ccccc2)c2ccccc2)c(O)n1] | (M + 1)⁺ 437 IC₅₀ 0.9 nM |
| Ex. 6 | (2S)-2-(5- (benzhydrylcarbamoyl)-4- hydroxypyrimidine-2- carboxamido)-3-(3aH-indo1- 3-yl)propanoic acid | 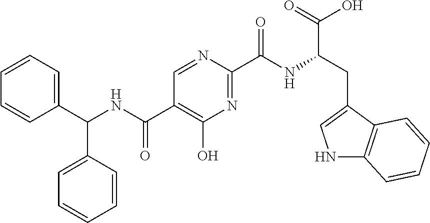 [SMILES: O=C(N[C@@H](Cc1c[nH]c2ccccc12)C(=O)O)c1ncc(C(=O)NC(c2ccccc2)c2ccccc2)c(O)n1] | (M + 1)⁺ 536 IC₅₀ 8.9 nM |
| Ex. 7 | (S)-3-(5- (benzhydrylcarbamoyl)-4- hydroxypyrimidine-2- carboxamido)-2- hydroxypropanoic acid | 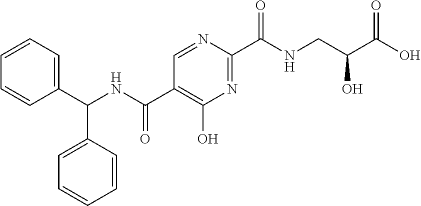 [SMILES: O=C(NC[C@H](O)C(=O)O)c1ncc(C(=O)NC(c2ccccc2)c2ccccc2)c(O)n1] | (M + 1)⁺ 437 IC₅₀ 1.4 nM |

### Example 8 and Example 9

### (R)-3-(5-(((4-Cyanophenyl)(phenyl methyl)carbamoyl)-4-hydroxypyrimidine-2-carboxamido)-2-hydroxypropanoic acid (Ex. 8) and (S)-3-(5-(((4-Cyanophenyl)(phenyl)methyl)carbamoyl)-4-hydroxypyrimidine-2-carboxamido)-2-hydroxypropanoic acid (Ex. 9)

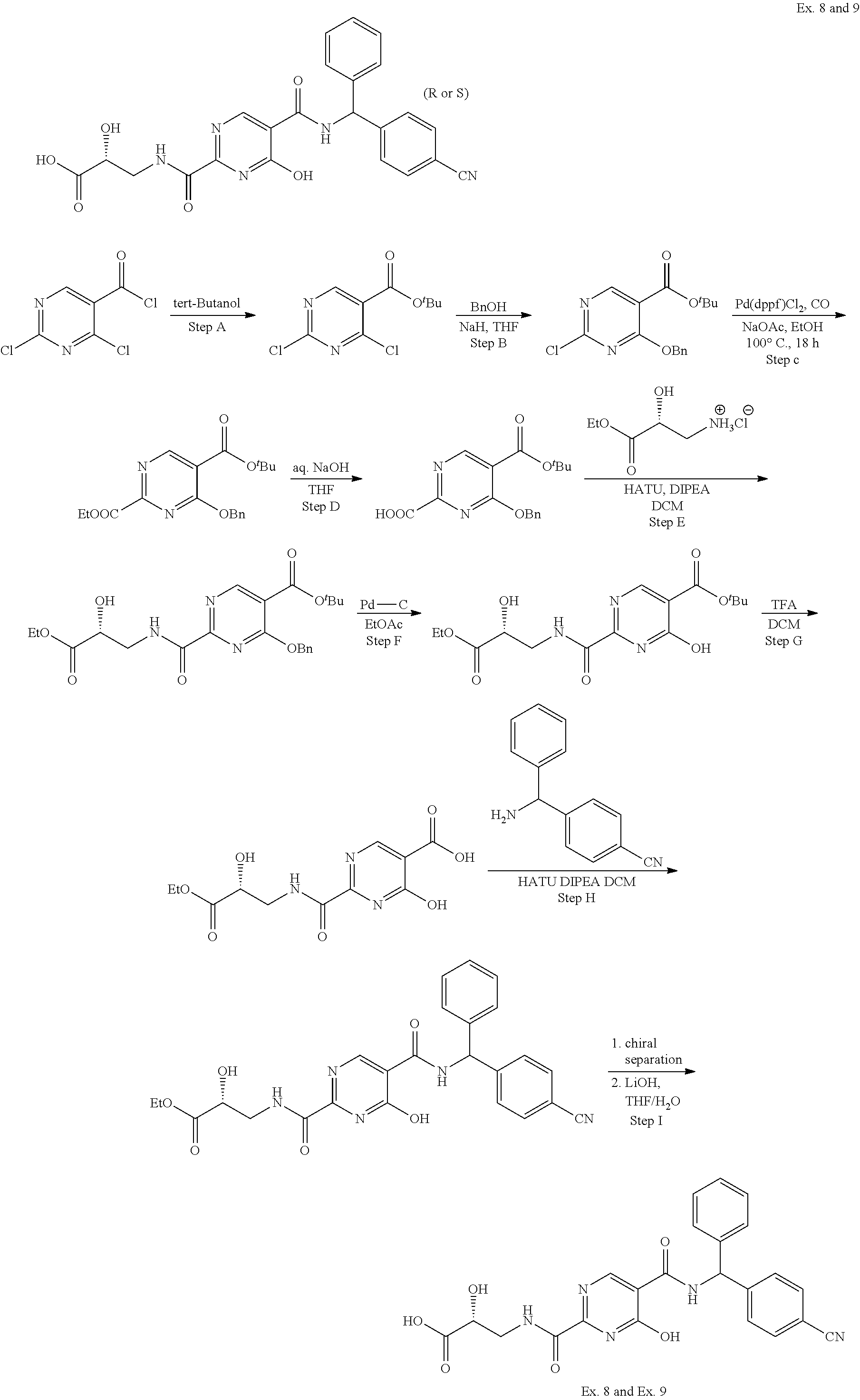

### Step A tert-Butyl 2,4-dichloropyrimidine-5-carboxylate

To a 250 mL round bottom flask equipped with magnetic stirrer was added tert-butanol (120 mL), followed by 2, 4-dichloropyrimidine-5-carbonyl chloride (30 g, 142 mmol). The reaction mixture was stirred at 30° C. for 16 hours. When TLC analysis showed that the reaction was complete, the reaction mixture was diluted with EtOAc (500 mL), and subsequently washed with water (300 mL), saturated aq. NaHCO₃ (200 mL), brine (200 mL). The organic layer was dried over Na₂SO₄ and concentrated under reduced pressure to afford tert-butyl 2, 4-dichloropyrimidine-5-carboxylate. ¹H NMR (CDCl₃, 400 MHz) δ 8.94 (s, 1H), 1.60 (s, 9H).

### Step B tert-Butyl 4-(benzyloxy)-2-chloropyrimidine-5-carboxylate

To a three neck flask charged with anhydrous THF (150 ml) was added NaH (4.1 g, 60% in paraffin oil, 102 mmol). To the above suspension, benzyl alcohol (9.6 ml, 93 mmol) was then added dropwise at 0° C. After the addition, the ice bath was removed and the reaction mixture was stirred at room temperature for 1 h. The resulting sodium benzoxide product was then transferred to a dry addition funnel. The sodium benzoxide mixture was added dropwise to a solution of tert-butyl 2,4-dichloropyrimidine-5-carboxylate (22.0 g, 88.4 mmol) in THF (250 mL) at 0° C. After stirring at 0° C. for 1 hour, LCMS analysis showed that the reaction was complete. Subsequently, saturated NH₄Cl (200 mL) was added to quench the reaction. The mixture was extracted with EtOAc (300 mL). The organic layer was washed with brine (200 mL), dried over Na₂SO₄ and concentrated under vacuum to afford crude product which was triturated with petroleum ether, and then followed by filtration to afford tert-butyl 4-(benzyloxy)-2-chloropyrimidine-5-carboxylate. ¹H NMR (CDCl₃, 400 MHz) δ 8.79 (s, 1H), 7.50-7.48 (m, 2H), 7.41-7.32 (m, 3H), 5.53 (s, 2H), 1.51 (s, 9H). LC/MS (m/z): 321 (M+H)⁺.

### Step C 5-tert-Butyl 2-ethyl 4-(benzyloxy)pyrimidine-2,5-dicarboxylate

To a 1 L stainless steel autoclave was added tert-butyl 4-(benzyloxy)-2-chloropyrimidine-5-carboxylate (7.0 g, 21.8 mmol), Pd(dppf)Cl₂ (1.6 g, 2.18 mmol), NaOAc (3.58 g, 43.6 mmol) and EtOH (300 mL). Then the air in the autoclave was replaced with carbon monoxide and the reaction mixture was stirred under 3.6 MPa at 70° C. for 18 hours. After cooling to room temperature, the reaction mixture was filtered through a pad of Celite and the filtrate was concentrated in vacuum to afford crude product that was purified by column chromatography on silica gel (eluted with petroleum ether/EtOAc=6:1-5:1) to afford 5-tert-butyl 2-ethyl 4-(benzyloxy)pyrimidine-2,5-dicarboxylate. (CDCl₃, 400 MHz) δ 9.01 (s, 1H), 7.55-7.53 (m, 2H), 7.38-7.32 (m, 3H), 5.61 (s, 2H), 4.50 (q, J=7.1 Hz, 2H), 1.52 (s, 9H), 1.46 (t, J=7.1 Hz, 3H). LC/MS (m/z): 359 (M+H)⁺.

### Step D 4-(Benzyloxy)-5-(tert-butoxycarbonyl)pyrimidine-2-carboxylic acid

To a solution of 5-tert-butyl 2-ethyl 4-(benzyloxy)pyrimidine-2,5-dicarboxylate (9.8 g, 27.4 mmol) in THF (500 mL) was added aqueous NaOH solution (1.15 g, 28.7 mmol in 80 mL H₂O) dropwise via an addition funnel at room temperature over a 40 min period. After the addition, the reaction mixture was stirred at room temperature for 10 min. When TLC testing showed that the reaction was complete, the reaction mixture was acidified to pH=3-4 with 5% HCl and then the mixture was extracted with DCM (300 mL). The organic layer was dried over Na₂SO₄ and concentrated under reduced pressure, then to the residue was added petroleum ether, and the precipitate was collected by suction to give the desired 4-(benzyloxy)-5-(tert-butoxycarbonyl)pyrimidine-2-carboxylic acid. ¹H NMR (CDCl₃, 400 MHz) δ 9.02 (s, 1H), 7.53-7.51 (m, 2H), 7.40-7.32 (m, 3H), 5.65 (s, 2H), 1.53 (s, 9H). LC/MS (m/z): 331 (M+H)⁺.

### Step E (R)-tert-butyl4-(benzyloxy)-2-((3-ethoxy-2-hydroxy-3-oxopropyl)carbamoyl)pyrimidine-5-carboxylate

To a 100 mL single-neck flask equipped with a stirring bar was added (R)-3-ethoxy-2-hydroxy-3-oxopropan-1-amine hydrochloride (205 mg, 1.211 mmol) and DCM (5 ml). To this solution were added DIPEA (0.423 ml, 2.422 mmol), 4-(benzyloxy)-5-(tert-butoxycarbonyl)pyrimidine-2-carboxylic acid (205 mg, 1.2 mmol) and HATU (460 mg, 1.211 mmol) at room temperature. The mixture was stirred for 16 h at room temperature and then partitioned between EtOAc (150 mL) and water (100 mL). The org. phase was dried over anhydrous sodium sulfate and concentrated under vacuum. The residue was purified by column chromatography on silica gel and eluted with Petroleum Ether/EtOAc(2:1-1:2) to afford (R)-tert-butyl 4-(benzyloxy)-2-((3-ethoxy-2-hydroxy-3-oxopropyl)carbamoyl)pyrimidine-5-carboxylate. LC/MS (m/z): 446 (M+H)⁺.

### Step F (R)-tert-butyl 2-((3-ethoxy-2-hydroxy-3-oxopropyl)carbamoyl)-4-hydroxy pyrimidine-5-carboxylate

To a solution of (R)-tert-butyl 4-(benzyloxy)-2-((3-ethoxy-2-hydroxy-3-oxopropyl)carbamoyl)pyrimidine-5-carboxylate (3.0 g, 6.7 mmol) in EtOAc (200 mL) was added 10% Pd/C (wet, 10%, 320 mg). The air was replaced with H₂ (1 atm). The reaction mixture was stirred at rt. When LCMS testing showed the reaction was complete, the reaction mixture was filtered through CELITE, and the filter cake washed with EtOAc. The filtrate was concentrated under reduced pressure to give crude (R)-tert-butyl 2-((3-ethoxy-2-hydroxy-3-oxopropyl)carbamoyl)-4-hydroxy pyrimidine-5-carboxylate, which was used without purification. LC/MS (m/z): 356 (M+H)⁺.

### Step G (R)-2-((3-ethoxy-2-hydroxy-3-oxopropyl)carbamoyl)-4-hydroxypyrimidine-5-carboxylic acid

(R)-tert-Butyl 2-((3-ethoxy-2-hydroxy-3-oxopropyl)carbamoyl)-4-hydroxypyrimidine-5-carboxylate (2.3 g, 6.5 mmol) was dissolved in TFA/DCM (1:1, 25 mL) and stirred for 3 hours. When TLC analysis indicated that the reaction was complete, the solvent was removed under reduced pressure. The residue was diluted with PhMe (60 ml) and concentrated again to afford the crude product which was used directly without further purification. LC/MS (m/z): 300 (M+H)⁺.

### Step H (2R)-ethyl 3-(5-(((4-cyanophenyl)(phenyl)methyl)carbamoyl)-4-hydroxy pyrimidine-2-carboxamido)-2-hydroxypropanoate

To a solution of (R)-2-((3-ethoxy-2-hydroxy-3-oxopropyl)carbamoyl)-4-hydroxyl pyrimidine-5-carboxylic acid (100 mg, 0.34 mmol) in DMF (5 mL) was added 4-(amino(phenyl)methyl)benzonitrile (140 mg, 0.68 mmol) and HATU (260 mg, 0.68 mmol) at room temperature, then DIPEA (130 mg, 1.0 mmol) was added at 0° C., and the reaction mixture was stirred at room temperature for 16 hours. The reaction was poured into 20 ml of water and extracted with EtOAc (4×10 mL). The combined organics were dried with Na₂SO₄ and the solvent was removed under reduced pressure. The residue was purified by prep. HPLC to afford (2R)-ethyl 3-(5-(((4-cyanophenyl)(phenyl)methyl)carbamoyl)-4-hydroxypyrimidine-2-carboxamido)-2-hydroxypropanoate. LC/MS (m/z): 490 (M+H)⁺.

### Step I (R)-3-(5-(((4-cyanophenyl)(phenyl)methyl)carbamoyl)-4-Hydroxypyrimidine-2-carboxamido)-2-hydroxypropanoic acid

The benzylhydryl stereoisomers of (2R)-ethyl 3-(5-(((4-cyanophenyl)(phenyl)methyl)carbamoyl)-4-hydroxypyrimidine-2-carboxamido)-2-hydroxypropanoate were separated by SFC (Chiralpak® AD-H 250*4.6 mm I.D., 5 um; 40% iPrOH (0.05% DEA) in CO2; 2.35 mL/min 220 nm) to give isomer 1 (the first peak) and isomer 2 (the second peak).

To a solution of isomer 1 of (R)-ethyl 2-hydroxy-3-(4-hydroxy-5-((2-(4-(trifluoromethyl)phenyl) propan-2-yl)carbamoyl)pyrimidine-2-carboxamido)propanoate (2.2 mg, 0.05 mmol) in THF (5 mL) was added aq. LiOH (10%, 0.42 mL, 1.0 mmol). The mixture was stirred at room temperature for 4 h. When TLC analysis showed that the reaction was complete, the mixture was concentrated, and the residue was diluted in water (2 mL) and acidified by aq. HCl (5%) to pH=5˜6. The mixture was dissolved in 1 ml of DMSO, and the mixture was purified by prep. HPLC to afford Example 8: (R)-2-hydroxy-3-(4-hydroxy-5-((2-(4-(trifluoromethyl)phenyl) propan-2-yl)carbamoyl)pyrimidine-2-carboxamido)propanoic acid. ¹H NMR (400 MHz, DMSO-d₆) δ 8.95 (s, 1H), 7.82-7.80 (d, J=8.0 Hz, 2H), 7.34-7.36 (m, 5H), 6.33-6.31 (d, J=8.0 Hz, 2H) 4.20-4.19 (m, 1H), 3.65-3.48 (m, 2H). LC/MS (m/z): 462 (M+H)⁺. Human HI-PHD2 IC₅₀: 6.8 nM.

Example 9 was prepared following an analogous procedure to that described in the above paragraph using isomer 2 of (R)-ethyl 2-hydroxy-3-(4-hydroxy-5-((2-(4-(trifluoromethyl)phenyl)propan-2-yl)carbamoyl)pyrimidine-2-carboxamido)propanoate (the second peak of Step I) and the appropriate starting materials. LC/MS (m/z): 462 (M+H)⁺. Human HI-PHD2 IC₅₀: 27.2 nM.

### Example 10

### (R)-2-Hydroxy-3-(4-hydroxy-5-(((S)-(4-methoxyphenyl) (phenyl)methyl)carbamoyl)pyrimidine-2-carboxamido)propanoic acid (Ex. 10)

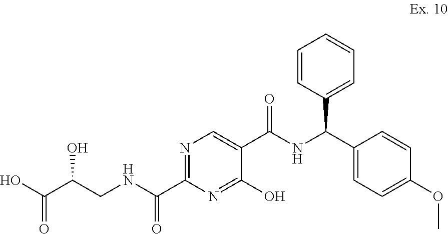 [SMILES: COc1ccc([C@@H](NC(=O)c2cnc(C(=O)NC[C@@H](O)C(=O)O)nc2O)c2ccccc2)cc1]

### Step 1 (R)-ethyl 2-hydroxy-3-(4-hydroxy-5-(((S)-(4-methoxyphenyl) (phenyl)methyl)carbamoyl)pyrimidine-2-carboxamido)propanoate

To a solution of (R)-2-((3-ethoxy-2-hydroxy-3-oxopropyl)carbamoyl)-4-hydroxypyrimidine-5-carboxylic acid (100 mg, 0.34 mmol) in DMF (5 mL) was added (S)-(4-methoxyphenyl)(phenyl)methanamine (140 mg, 0.68 mmol) and HATU (260 mg, 0.68 mmol) at room temperature. DIPEA (130 mg, 1.0 mmol) at 0° C. was added and the reaction mixture was stirred at room temperature for 16 hours. The reaction was poured into 20 ml of water and extracted with EtOAc (4×10 mL). The combined organics were dried over Na₂SO₄ and the solvent was removed under reduced pressure. The residue was purified by prep. HPLC to afford (R)-ethyl 2-hydroxy-3-(4-hydroxy-5-(((S)-(4-methoxyphenyl)(phenyl)methyl)carbamoyl)pyrimidine-2-carboxamido)propanoate. LC/MS (m/z): 495 (M+H)⁺.

### Step 2 (R)-2-hydroxy-3-(4-hydroxy-5-(((S)-(4-methoxyphenyl)(phenyl)methyl) carbamoyl)pyrimidine-2-carboxamido)propanoic acid

To a solution of (2S)-ethyl 3-(5-(((4-cyanophenyl)(phenyl)methyl)carbamoyl)-4-hydroxypyrimidine-2-carboxamido)-2-hydroxypropanoate (10 mg, 0.12 mmol) in THF (5 mL) was added aq. LiOH (10%, 0.42 mL, 1.0 mmol). The mixture was stirred at room temperature for 4 h. When TLC testing showed that the reaction was complete, the mixture was concentrated, and the residue was diluted in water (2 mL) and acidified by aq. HCl (5%) to pH=5˜6. The mixture was dissolved in 1 mL of DMSO, and purified by prep. HPLC to afford (R)-2-hydroxy-3-(4-hydroxy-5-(((S)-(4-methoxyphenyl)(phenyl)methyl)carbamoyl)pyrimidine-2-carboxamido)propanoic acid. ¹H NMR (DMSO-d₆, 400 MHz) δ 8.91 (brs, 1H), 8.14-8.13 (m, 1H), 7.59-7.57 (d, J=8.0 Hz, 1H), 7.41-7.32 (m, 6H), 6.80-6.78 (d, J=8.0 Hz, 1H) 6.25-6.23 (m, 1H), 4.20-4.19 (m, 1H), 3.79 (s., 3H), 3.65-3.48 (m, 2H). LC/MS (m/z): 467 (M+H)⁺. Human HI-PHD2 IC₅₀: 6.8 nM.

Examples 11 and 12 in Table 4 were prepared following analogous procedures to those described in Example 10 by using (R)-2-((3-ethoxy-2-hydroxy-3-oxopropyl)carbamoyl)-4-hydroxypyrimidine-5-carboxylic acid (Example 8, Step G) and the appropriate starting materials.

TABLE 4

| | | | MS m/z |
|---|---|---|---|
| | | | (M + 1)⁺ and |
| Example | | | human HIF- |
| Number | Name | Structure | PHD2 IC₅₀ |
| Ex. 11 | (R)-2-hydroxy-3-(4-hydroxy- 5-((2-(4-(trifluoromethyl) phenyl)propan-2- yl)carbamoyl)pyrimidine-2- carboxamido)propanoic acid | 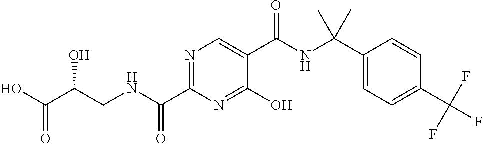 [SMILES: CC(C)(NC(=O)c1cnc(C(=O)NC[C@@H](O)C(=O)O)nc1O)c1ccc(C(F)(F)F)cc1] | (M + 1)+ 457 IC50 1.5 nM |
| Ex. 12 | (R)-2-hydroxy-3-(4-hydroxy- 5-(((R)-(4-methoxyphenyl) (phenyl)methyl)carbamoyl) pyrimidine-2- carboxamido)propanoic acid | 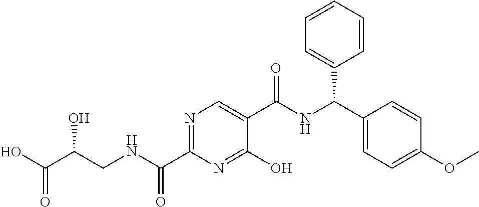 [SMILES: COc1ccc([C@H](NC(=O)c2cnc(C(=O)NC[C@@H](O)C(=O)O)nc2O)c2ccccc2)cc1] | (M + 1)+ 467 IC50 8.7 nM |

### Example 13

### 3-(5-((Bis(4-chlorophenyl)methyl)carbamoyl)-hydroxypyrimidine-2-carboxamido-methylpropanoic acid (Ex. 13′)

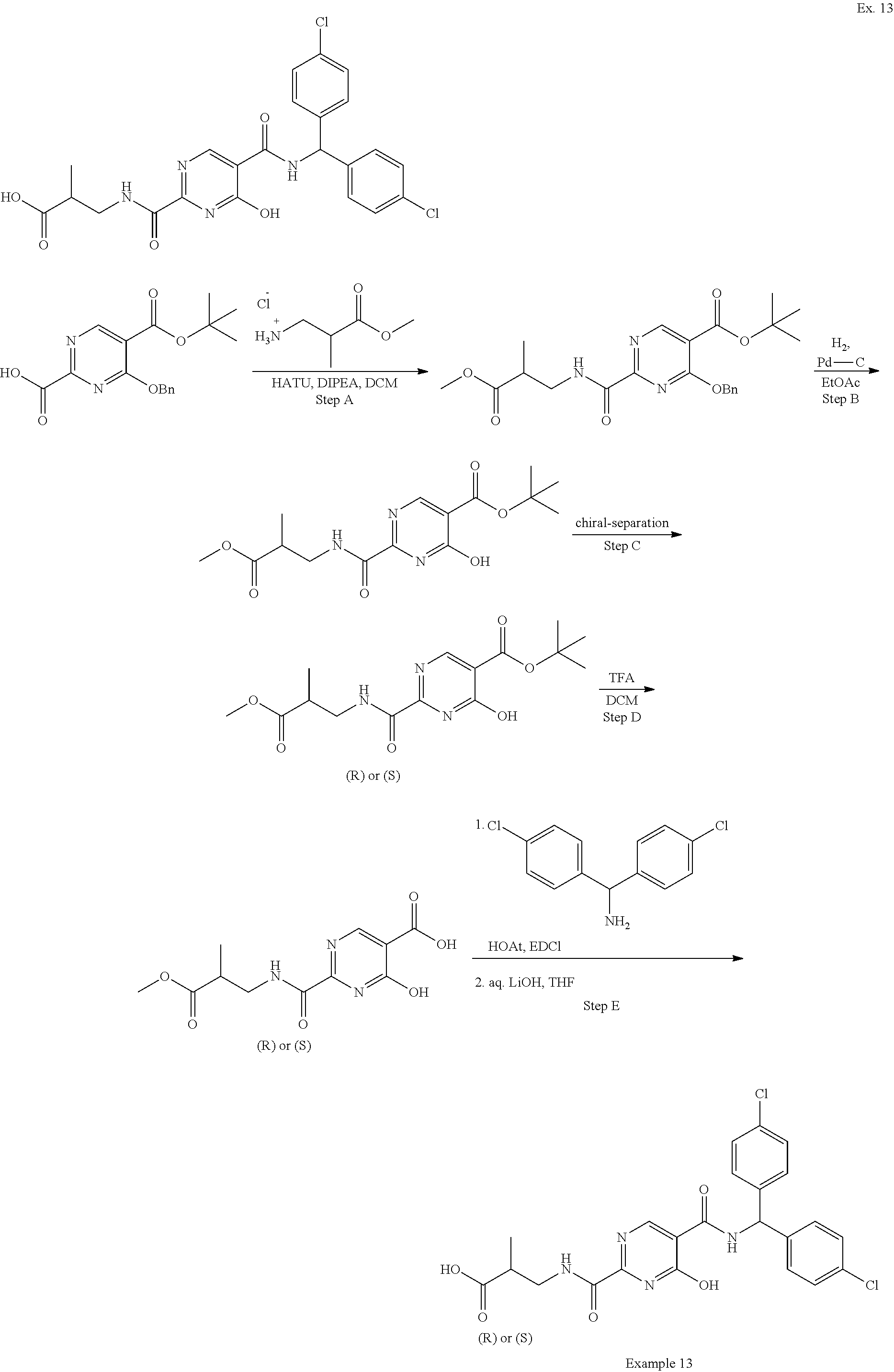

### Step A tert-Butyl 4-(benzyloxy)-2-((3-methoxy-2-methyl-3-oxopropyl)carbamoyl)pyrimidine-5-carboxylate

To a solution of 4-(benzyloxy)-5-(tert-butoxycarbonyl)pyrimidine-2-carboxylic acid (4.0 g, 12 mmol) in DCM (100 mL) was added 3-methoxy-2-methyl-3-oxopropan-1-aminium chloride (2.1 g, 13 mmol), DIPEA (7.8 g, 61 mmol) and HATU (9.2 g, 24 mmol). The reaction mixture was stirred at room temperature for 16 hours. When LCMS analysis showed the reaction was complete, the mixture was concentrated in vacuum. The residue was partitioned between water (50 mL) and EtOAc (50 mL), and the aq. phase was extracted with EtOAc (50 mL×2). The combined organic layers were washed with water and a.q. HCl (5%), dried over Na₂SO₄ and concentrated in vacuum. The residue was purified by CombiFlash® (eluted with petroleum ether:EtOAc=5:1-3:1) to afford tert-butyl 4-(benzyloxy)-2-((3-methoxy-2-methyl-3-oxopropyl)carbamoyl)pyrimidine-5-carboxylate. ¹H NMR (CDCl₃, 400 MHz) δ: 8.99 (s, 1H), 8.36 (br s, 1H), 7.55-7.53 (m, 2H), 7.41-7.33 (m, 3H), 5.65 (s, 2H), 3.78-3.76 (m, 1H), 3.73 (s, 3H), 3.60-3.55 (m, 1H), 2.88-2.83 (m, 1H), 1.55 (s, 9H), 1.27 (d, J=7.2 Hz, 3H).

### Step B tert-Butyl 4-hydroxy-2-((3-methoxy-2-methyl-3-oxopropyl)carbamoyl)pyrimidine-5-carboxylate

To a solution of tert-butyl 4-(benzyloxy)-2-((3-methoxy-2-methyl-3-oxopropyl)carbamoyl)pyrimidine-5-carboxylate (5.0 g, 12 mmol) in EtOAc (50 mL) was added Pd/C (wet, 5%, 0.5 g). The mixture was stirred under hydrogenation atmosphere for 16 h. When TLC analysis showed that the reaction was complete, the reaction mixture was filtered through a pad of Celite. The filtrate was concentrated under reduced pressure to afford tert-butyl4-hydroxy-2-((3-methoxy-2-methyl-3-oxopropyl)carbamoyl)pyrimidine-5-carboxylate. ¹H NMR (CDCl₃, 400 MHz) δ: 8.58 (br s, 1H), 8.23 (br s, 1H), 3.74 (s, 3H), 3.70-3.66 (m, 1H), 3.65-3.54 (m, 1H), 2.83-2.78 (m, 1H), 1.58 (s, 9H), 1.25 (d, J=6.8 Hz, 3H).

### Step C Isomer 1 of tert-butyl 4-hydroxy-2-((3-methoxy-2-methyl-3-oxopropyl) carbamoyl)pyrimidine-5-carboxylate

The two chiral isomers of tert-butyl 4-hydroxy-2-((3-methoxy-2-methyl-3-oxopropyl)carbamoyl)pyrimidine-5-carboxylate (4.0 g) were resolved through chiral SFC (SFC Condition: Column: Chiralpak® AD-H 250×4.6 mm I.D., 5 um Mobile phase: 40% iso-propanol (0.05% DEA) in CO₂ Flow rate: 2.35 mL/min Wavelength: 220 nm) to afford:

Isomer 1 (first peak, RT=2.508 min) of tert-butyl 4-hydroxy-2-((3-methoxy-2-methyl-3-oxopropyl)carbamoyl)pyrimidine-5-carboxylate; and
Isomer 2 (second peak, RT=2.968 min)

### Step D 4-hydroxy-2-((3-methoxy-2-methyl-3-oxopropyl)carbamoyl)pyrimidine-5-carboxylic acid

To a solution of tert-butyl4-hydroxy-2-((3-methoxy-2-methyl-3-oxopropyl) carbamoyl)pyrimidine-5-carboxylate (Isomer 1 of Step C, 0.48 g, 1.4 mmol) in DCM (20 mL) was added TFA (20 mL). The mixture was stirred at room temperature for 1 hour. When LCMS testing showed that the reaction was complete, the mixture was concentrated under reduced pressure to afford 4-hydroxy-2-((3-methoxy-2-methyl-3-oxopropyl)carbamoyl)pyrimidine-5-carboxylic acid. ¹H NMR (CDCl₃, 400 MHz) δ: 8.95 (br s, 1H), 8.36 (br s, 1H), 3.73 (s, 3H), 3.70-3.67 (m, 1H), 3.63-3.58 (m, 1H), 2.84-2.79 (m, 1H), 1.26 (d, J=6.8 Hz, 3H).

### Step E 3-(5-((bis(4-chlorophenyl)methyl)carbamoyl)-4-hydroxypyrimidine-2-carboxamido)-2-methylpropanoic acid

To a solution of 4-hydroxy-2-((3-methoxy-2-methyl-3-oxopropyl)carbamoyl)pyrimidine-5-carboxylic acid (100 mg, 0.36 mmol) in DMF (5 mL) was added bis(4-chlorophenyl) methanamine (210 mg, 0.72 mmol) and HOAt (98 mg, 0.72 mmol) at room temperature. Subsequently, EDCI (100 mg, 0.54 mmol) was added at 0° C. The reaction mixture was stirred at room temperature for 16 hours. The mixture was concentrated in vacuum to remove DMF. The residue was purified by prep. TLC (eluted with petroleum ether:EtOAc=1:5) to afford methyl 5-((bis(4-chlorophenyl)methyl)carbamoyl)-4-hydroxypyrimidine-2-carboxamido)-2-methylpropanoate. ¹H NMR (400 MHz, CDCl₃) δ: 9.83 (br s, 1H), 8.96 (s, 1H), 8.26 (s, 1H), 7.32-7.30 (m, 4H), 7.34-7.22 (m, 4H), 6.36 (d, J=8.0 Hz, 1H), 3.75 (s, 3H), 3.68-3.70 (m, 2H), 2.81-2.79 (m, 1H), 1.25 (d, J=8.0 Hz, 3H).

To a solution of methyl 3-(5-((bis(4-chlorophenyl)methyl)carbamoyl)-4-hydroxypyrimidine-2-carboxamido)-2-methylpropanoate (100 mg, 0.18 mmol) in THF (10 mL) was added aq. LiOH (10%, 2 mL, 2 mmol). The mixture was stirred at room temperature for 16 h. When TLC sampling showed that the reaction was complete, the mixture was concentrated, and the residue was diluted in water (2 mL) and acidified by aq. HCl (5%) to pH=1-2. The precipitate was collected by suction to afford the title compound. ¹H NMR (DMSO-d₆, 400 MHz) δ: 13.53 (br s, 1H), 12.36 (br s, 1H), 10.47 (br s, 1H), 9.13 (t, J=6.0 Hz, 1H), 8.43 (br s, 1H), 7.48-7.36 (m, 4H), 7.35-7.24 (m, 4H), 6.26 (d, J=7.8 Hz, 1H), 3.50-3.42 (m, 1H), 3.28-3.24 (m, 1H), 2.74-2.65 (m, 1H), 1.03 (d, J=7.3 Hz, 3H). LC/MS (m/z): 503 (M+1)⁺. Human HIF-PHD2 IC₅₀: 4.88 nM.

Examples 14 through 16 in Table 5 were prepared following analogous procedures to those described for Example 13 using isomer 1 (the first peak) and the appropriate starting materials.

TABLE 5

| | | | MS m/z |
|---|---|---|---|
| | | | (M + 1)⁺ and |
| Example | | | human HIF- |
| Number | Name | Structure | PHD2 IC₅₀ |
| Ex. 14 | (R) or (S)-3-(4- hydroxy-5-(((4- methoxyphenyl)(6- methoxyquinolin-2- yl)methyl)carbamoyl) pyrimidine-2- carboxamido)-2- methylpropanoic acid | 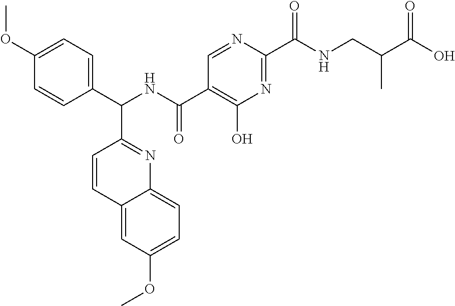 [SMILES: COc1ccc(C(NC(=O)c2cnc(C(=O)NCC(C)C(=O)O)nc2O)c2ccc3cc(OC)ccc3n2)cc1] | (M + 1)⁺ 546 IC₅₀ 16 nM |
| Ex. 15 | (R) or (S)-3-(5-((bis(4- methoxyphenyl)methy) carbamoyl)-4- hydroxypyrimidine-2- carboxamido)-2- methylpropanoic acid | 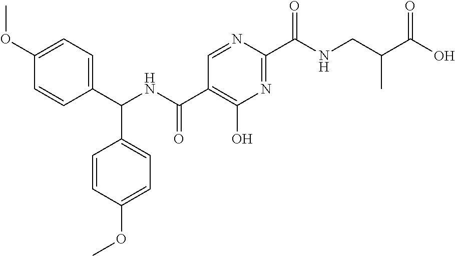 [SMILES: COc1ccc(C(NC(=O)c2cnc(C(=O)NCC(C)C(=O)O)nc2O)c2ccc(OC)cc2)cc1] | (M + 1)⁺ 495 IC₅₀ 10 nM |
| Ex. 16 | (R) or (S)-3-(5-((1-(4- bromophenyl)cyclopropyl) carbamoyl)-4- hydroxypyrimidine-2- carboxamido)-2- methylpropanoic acid | 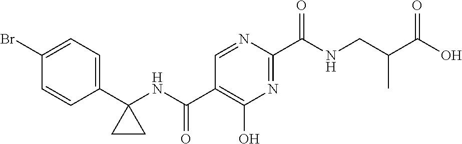 [SMILES: CC(CNC(=O)c1ncc(C(=O)NC2(c3ccc(Br)cc3)CC2)c(O)n1)C(=O)O] | (M + 1)⁺ 463 IC₅₀ 1.3 nM |

Examples 17 through 19 in Table 6 were prepared following an analogous synthesis route to that describe for Example 13 by using isomer 2 (the second peak) and the appropriate starting materials.

TABLE 6

| | | | MS m/z |
|---|---|---|---|
| | | | (M + 1)⁺ and |
| Example | | | human HIF- |
| Number | Name | Structure | PHD2 IC₅₀ |
| Ex. 17 | (R) or (S)-3-(5-((1- (4-bromophenyl) cyclopropyl) carbamoyl)-4- hydroxypyrimidine- 2-carboxamido)-2- methylpropanoic acid | 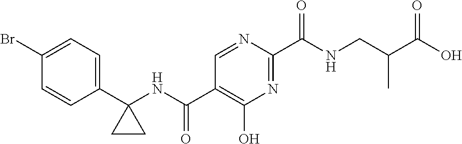 [SMILES: CC(CNC(=O)c1ncc(C(=O)NC2(c3ccc(Br)cc3)CC2)c(O)n1)C(=O)O] | (M + 1)⁺ 463 IC₅₀: 0.8 nM |
| Ex. 18 | (R) or (S)-3-(5- ((bis(4- methoxyphenyl) methyl) carbamoyl)-4- hydroxypyrimidine- 2-carboxamido)-2- methylpropanoic acid | 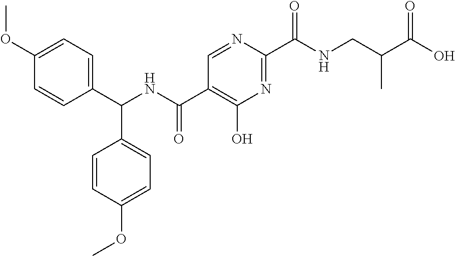 [SMILES: COc1ccc(C(NC(=O)c2cnc(C(=O)NCC(C)C(=O)O)nc2O)c2ccc(OC)cc2)cc1] | (M + 1)⁺ 495 IC₅₀ 15 nM |
| Ex. 19 | (R) or (S)-3-(4- hydroxy-5-((2-(6- (4-methoxyphenyl) pyridin- 3-yl)propan-2- yl)carbamoyl) pyrimidine- 2-carboxamido)-2- methylpropanoic acid | 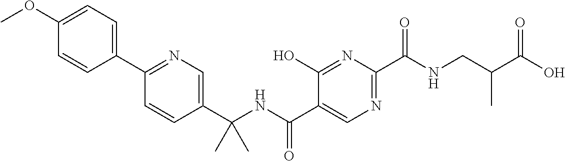 [SMILES: COc1ccc(-c2ccc(C(C)(C)NC(=O)c3cnc(C(=O)NCC(C)C(=O)O)nc3O)cn2)cc1] | (M + 1)⁺ 494 IC₅₀ 2.8 nM |

### Example 20

### (R) or (S)-3-(4-hydroxy-5-((2-(4′-(trifluoromethyl)-[1,1′-biphenyl]-4-yl)propan-2-yl)carbamoyl)pyrimidine-2-carboxamido)-2-methylpropanoic acid

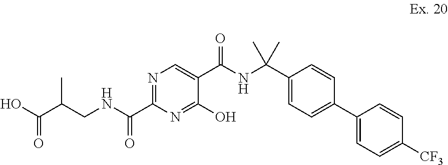 [SMILES: CC(CNC(=O)c1ncc(C(=O)NC(C)(C)c2ccc(-c3ccc(C(F)(F)F)cc3)cc2)c(O)n1)C(=O)O]

To a solution of 2-(4′-(trifluoromethyl)-[1,1′-biphenyl]-4-yl)propan-2-amine (20 mg, 0.07 mmol) in DMF (2 mL) was added acid from isomer 2 of 2-((3-ethoxy-2-methyl-3-oxopropyl)carbamoyl)-4-hydroxypyrimidine-5-carboxylic acid (Example 13 Step C, 32 mg, 0.11 mmol) and HOAt (20 mg, 0.14 mmol) at room temperature, then followed by EDCI (21 mg, 0.11 mmol) at 0° C., and the mixture was stirred at room temperature for 16 h. After concentration under vacuum to remove DMF, the residue was purified by prep. TLC (eluted with petroleum ether:EtOAc=1:5) to afford ethyl3-(4-hydroxy-5-((2-(4′-(trifluoromethyl)-[1,1′-biphenyl]-4-yl)propan-2-yl)carbamoyl)pyrimidine-2-carboxamido)-2-methylpropanoate. LC/MS (m/z): 559 (M+1)⁺.

To a solution of ethyl 3-(4-hydroxy-5-((2-(4′-(trifluoromethyl)-[1,1′-biphenyl]-4-yl) propan-2-yl)carbamoyl)pyrimidine-2-carboxamido)-2-methylpropanoate (30 mg, 0.05 mmol) in THF (2 mL) was added aq. LiOH (10%, 0.5 mL, 2.0 mmol). The mixture was stirred at room temperature for 6 h. When TLC analysis showed that the reaction was complete, the mixture was concentrated, and the residue was diluted with water (2 mL) and acidified with aq. HCl (5%) to pH=4-5. The precipitate was collected by suction to afford to 3-(4-hydroxy-5-((2-(4′-(trifluoromethyl)-[1,1′-biphenyl]-4-yl)propan-2-yl)carbamoyl)pyrimidine-2-carboxamido)-2-methylpropanoic acid. ¹H NMR (400 MHz, DMSO-d₆) δ: 13.38 (br s, 1H), 12.36 (br s, 1H), 10.17 (br s, 1H), 9.14 (t, J=6.0 Hz, 1H), 8.26 (br s, 1H), 7.85 (d, J=8.4 Hz, 2H), 7.77 (d, J=8.4 Hz, 2H), 7.65 (d, J=8.4 Hz, 2H), 7.47 (d, J=8.8 Hz, 2H), 3.50-3.44 (m, 1H), 3.27-3.25 (m, 1H), 2.73-2.68 (m, 1H), 1.69 (s, 6H), 1.04 (d, J=7.2 Hz, 3H). LC/MS (m/z): 531 (M+1)⁺. Human HIF-PHD2 IC₅₀: 8.1 nM.

Examples 2 through 33, in Table 7 were prepared following an analogous synthesis scheme to that described for Example 20 and by using the appropriate starting materials.

TABLE 7

| | | | MS m/z |
|---|---|---|---|
| | | | (M + 1)⁺ and |
| Example | | | human HIF- |
| Number | Name | Structure | PHD2 IC₅₀ |
| Ex. 21 | (R) or (S)-2-((2- carboxypropyl) carbamoyl)- 6-hydroxy-5-((2-(4- (isoquinolin-5- yl)phenyl)propan-2- yl)carbamoyl) pyrimidin-1- ium formate | 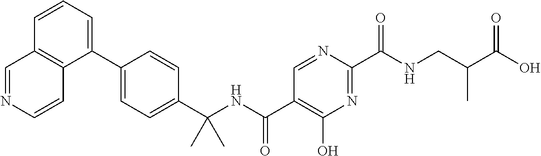 [SMILES: CC(CNC(=O)c1ncc(C(=O)NC(C)(C)c2ccc(-c3cccc4cnccc34)cc2)c(O)n1)C(=O)O] | (M + 1)⁺ 514 IC₅₀ 4.8 nM |
| Ex. 22 | (R) or (S)-2-((2- carboxypropyl) carbamoyl)- 6-hydroxy-5-((2-(4- (pyridin-3- yl)phenyl)propan-2- yl)carbamoyl) pyrimidin-1- ium formate | 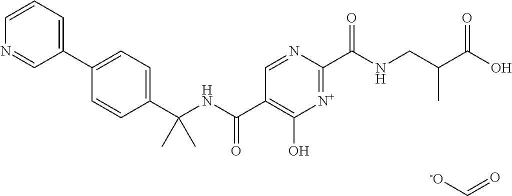 | (M + 1)⁺ 464 IC₅₀ 3.0 nM |
| Ex. 23 | (R) or (S)-3-(5-((2-(4′- cyano-2′-methyl-[1,1′- biphenyl]-4-yl)propan-2- yl)carbamoyl)-4- hydroxypyrimidine-2- carboxamido)-2- methylpropanoic acid | 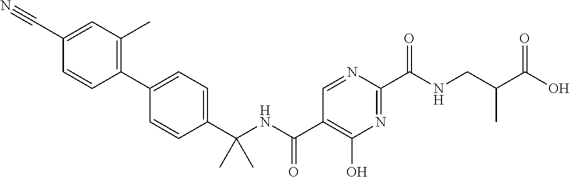 [SMILES: Cc1cc(C#N)ccc1-c1ccc(C(C)(C)NC(=O)c2cnc(C(=O)NCC(C)C(=O)O)nc2O)cc1] | (M + 1)⁺ 502 IC₅₀ 12 nM |
| Ex. 24 | (R) or (S)-3-(5-((2-(4′- fluoro-[1,1′-biphenyl]-4- yl)propan-2- yl)carbamoyl)-4- hydroxypyrimidine-2- carboxamido)-2- methylpropanoic acid | 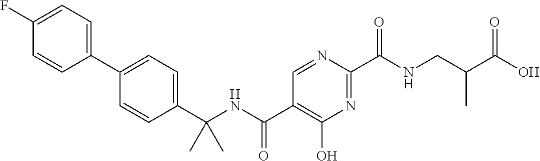 [SMILES: CC(CNC(=O)c1ncc(C(=O)NC(C)(C)c2ccc(-c3ccc(F)cc3)cc2)c(O)n1)C(=O)O] | (M + 1)⁺ 481 IC₅₀ 4.8 nM |
| Ex. 25 | (R) or (S)-3-(4-hydroxy-5- ((2-(4′-(trifluoromethoxy)- [1,1′-biphenyl]-4- yl)propan-2- yl)carbamoyl)pyrimidine- 2-carboxamido)-2- methylpropanoic acid | 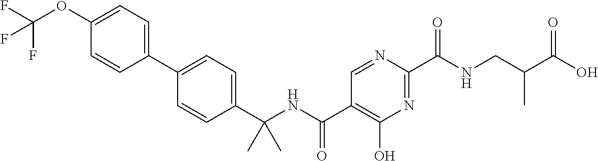 [SMILES: CC(CNC(=O)c1ncc(C(=O)NC(C)(C)c2ccc(-c3ccc(OC(F)(F)F)cc3)cc2)c(O)n1)C(=O)O] | (M + 1)⁺ 547 IC₅₀ 13 nM |
| Ex. 26 | (R) or (S)-2-((2- carboxypropyl) carbamoyl)- 6-hydroxy-5-((2-(4-(6- methoxypyridin-3- yl)phenyl)propan-2- yl)carbamoyl) pyrimidin-1- ium chloride | 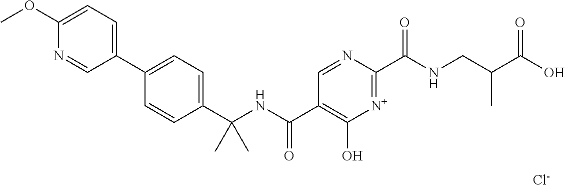 [SMILES: COc1ccc(-c2ccc(C(C)(C)NC(=O)c3cnc(C(=O)NCC(C)C(=O)O)[nH+]c3O)cc2)cn1 ; [Cl-]] | (M + 1)⁺ 494 IC₅₀ 3.4 nM |
| Ex. 27 | (R) or (S)-5-((2-(4-(1H- pyrazol-3- yl)phenyl)propan-2- yl)carbamoyl)-2-((2- carboxypropyl) carbamoyl)- 6-hydroxypyrimidin-1- ium formate | 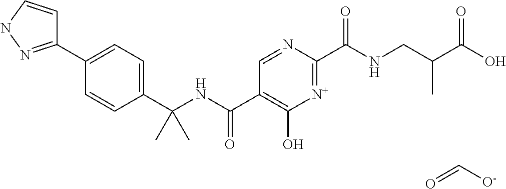 | (M + 1)⁺ 453 IC₅₀ 5.8 nM |
| Ex. 28 | (R) or (S)-3-(5-((2-(4′- chloro-[1,1′-biphenyl]-4- yl)propan-2- yl)carbamoyl)-4- hydroxypyrimidine-2- carboxamido)-2- methylpropanoic acid | 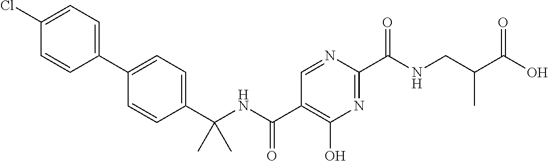 [SMILES: CC(CNC(=O)c1ncc(C(=O)NC(C)(C)c2ccc(-c3ccc(Cl)cc3)cc2)c(O)n1)C(=O)O] | (M + 1)⁺ 497 IC₅₀ 5.9 nM |
| Ex. 29 | (R) or (S)-3-(4-hydroxy-5- ((1-(4′-(trifluoromethyl)- [1,1′-biphenyl]-4- yl)cyclopropyl) carbamoyl)pyrimidine-2- carboxamido)-2-methyl propanoic acid | 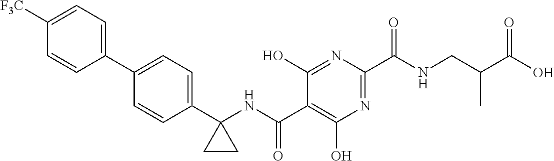 | (M + 1)⁺ 529 IC₅₀ 8.0 nM |
| Ex. 30 | (R) or (S)-3-(5-((1-(4′- chloro-[1,1′-biphenyl]-4- yl)cyclopropyl) carbamoyl)-4- hydroxypyrimidine-2- carboxamido)-2- methylpropanoic acid | 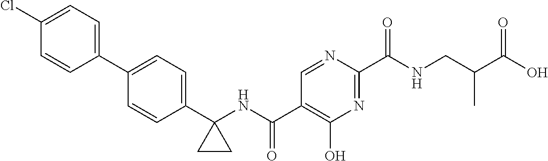 [SMILES: CC(CNC(=O)c1ncc(C(=O)NC2(c3ccc(-c4ccc(Cl)cc4)cc3)CC2)c(O)n1)C(=O)O] | (M + H)⁺ 495 IC₅₀ 13 nM |
| Ex. 31 | (R) or (S)-2-((2- carboxypropyl) carbamoyl)- 6-hydroxy-5-((1-(4- (isoquinolin-5- yl)phenyl)cyclopropyl) carbamoyl)pyrimidin-1- ium formate | 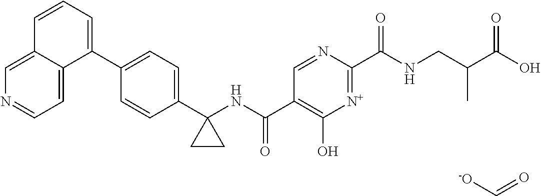 | (M + H)⁺ 512 IC₅₀ 10 nM |
| Ex. 32 | (R) or (S)-3-(5-((1-(4′- fluoro-[1,1′-biphenyl]-4- yl)cyclopropyl) carbamoyl)-4- hydroxypyrimidine-2- carboxamido)-2- methylpropanoic acid | 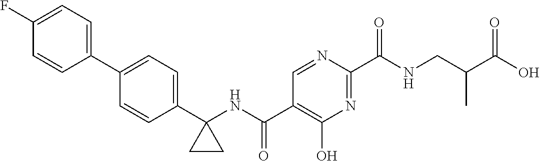 [SMILES: CC(CNC(=O)c1ncc(C(=O)NC2(c3ccc(-c4ccc(F)cc4)cc3)CC2)c(O)n1)C(=O)O] | (M + H)⁺ 479 IC₅₀ 12 nM |
| Ex. 33 | (R) or (S)-2-((2- carboxypropyl) carbamoyl)- 6-hydroxy-5-((1-(4-(6- methoxypyridin-3- yl)phenyl)cyclopropyl) carbamoyl)pyrimidin-1- ium chloride | 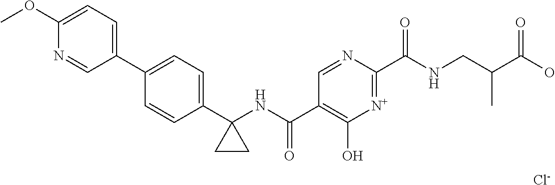 [SMILES: COc1ccc(-c2ccc(C3(NC(=O)c4cnc(C(=O)NCC(C)C(=O)O)[nH+]c4O)CC3)cc2)cn1 ; [Cl-]] | (M + H)⁺ 492 IC₅₀ 4.3 nM |

### Example 34

### (R) or (S)-3-(4-hydroxy-5-(((4-methoxyphenyl)(6-(methylamino)pyridin-3-yl)methyl)carbamoyl)pyrimidine-2-carboxamido)propanoic acid (Ex. 34)

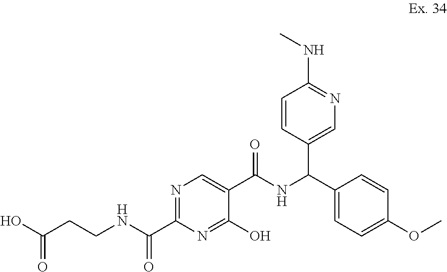

To a solution of 2-((3-ethoxy-3-oxopropyl)carbamoyl)-4-hydroxypyrimidine-5-carboxylic acid (100 mg, 0.36 mmol) in DMF (5 mL) was added 5-(amino(4-methoxyphenyl)methyl)-N-methylpyridin-2-amine (260 mg, 0.72 mmol) and HOAt (98 mg, 0.72 mmol) at room temperature. Subsequently, EDCI (100 mg, 0.54 mmol) was added to the reaction mixture at 0° C. The reaction mixture was stirred at room temperature for 16 hours. The mixture was then concentrated in vacuum to remove DMF. The residue was purified by prep. HPLC to afford ethyl 3-(4-hydroxy-5-(((4-methoxyphenyl)(6-(methylamino)pyridin-3-yl)methyl)carbamoyl)pyrimidine-2-carboxamido)propanoate. LC/MS (m/z): 509 (M+H)⁺.

To a solution of ethyl 3-(4-hydroxy-5-(((4-methoxyphenyl) (6-(methylamino)pyridin-3-yl)methyl)carbamoyl)pyrimidine-2-carboxamido) propanoate (70 mg, 0.14 mmol) in MeOH (5 mL) was added aq. NaOH (5%, 1 mL, 1 mmol). The mixture was stirred at room temperature for 6 h. When TLC analysis showed that the reaction was complete, the mixture was concentrated, and the residue was purified by prep. HPLC to afford 3-(4-hydroxy-5-(((4-methoxyphenyl) (6-(methylamino)pyridin-3-yl)methyl)carbamoyl)pyrimidine-2-carboxamido)propanoic acid. ¹H NMR (DMSO-d₆. 400 MHz) δ 12.75 (br s, 1H), 10.21 (s, 1H), 9.17 (t, J=6.0 Hz, 1H), 8.49 (s, 1H), 7.77 (s, 2H), 7.07 (d, J=10.0 Hz, 2H), 7.29 (d, J=8.4 Hz, 2H), 6.96 (d, J=8.4 Hz, 1H), 6.19 (d, J=8.0 Hz, 1H), 3.74 (s, 3H), 3.47 (q, J=7.2 Hz, 2H), 2.96 (s, 3H), 2.54 (t, J=8.4 Hz, 2H). LC/MS (m/z): 481 (M+H)⁺. Human HIF-PHD2 IC₅₀: 0.58 nM.

Examples 35 through 49 in Table 8 were prepared following an analogous procedure to that described in Example 34 using 4-hydroxy-2-((3-methoxy-3-oxopropyl)carbamoyl)pyrimidine-5-carboxylic and by using appropriate starting materials.

TABLE 8

| | | | MS m/z |
|---|---|---|---|
| | | | (M + 1)⁺ and |
| Example | | | human HIF- |
| Number | Name | Structure | PHD2 IC₅₀ |
| Ex. 35 | (R)-3-(5-(((4- chlorophenyl)(4- methoxyphenyl)methyl) carbamoyl)-4- hydroxypyrimidine-2- carboxamido)propanoic acid | 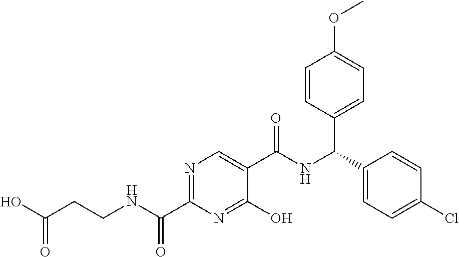 [SMILES: COc1ccc([C@@H](NC(=O)c2cnc(C(=O)NCCC(=O)O)nc2O)c2ccc(Cl)cc2)cc1] | (M + 1)⁺ 485 IC₅₀ 0.9 nM |
| Ex. 36 | (S)-3-(4-hydroxy-5-(((4- methoxyphenyl)(phenyl) methyl)carbamoyl) pyrimidine-2- carboxamido)propanoic acid | 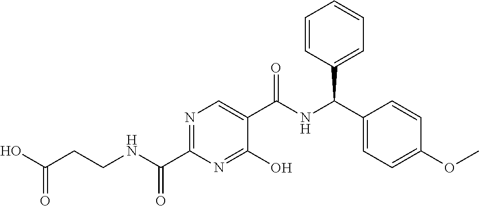 [SMILES: COc1ccc([C@@H](NC(=O)c2cnc(C(=O)NCCC(=O)O)nc2O)c2ccccc2)cc1] | (M + 1)⁺ 451 IC50 2.7 nM |
| Ex. 37 | (R)-3-(5-(((4- chlorophenyl)(4- methoxyphenyl)methyl) carbamoyl)-4- hydroxypyrimidine-2- carboxamido)propanoic acid | 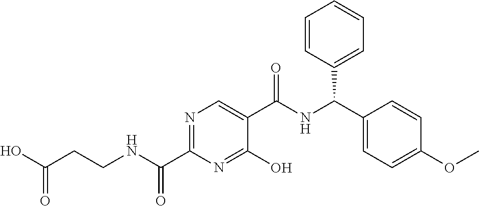 [SMILES: COc1ccc([C@H](NC(=O)c2cnc(C(=O)NCCC(=O)O)nc2O)c2ccccc2)cc1] | (M + 1)⁺ 451 IC50 2.4 nM |
| Ex. 38 | 3-(5-(((4-chlorophenyl)(4- methoxyphenyl)methyl) carbamoyl)-4- hydroxypyrimidine-2- carboxamido)propanoic acid | 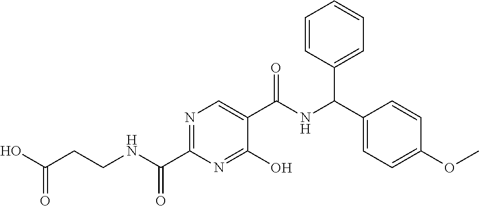 [SMILES: COc1ccc(C(NC(=O)c2cnc(C(=O)NCCC(=O)O)nc2O)c2ccccc2)cc1] | (M + 1)⁺ 451 IC50 0.9 nM |
| Ex. 39 | 3-(5-(((2,3- dihydrobenzofuran-5-yl)(4- methoxyphenyl)methyl) carbamoyl)-4- hydroxypyrimidine-2- carboxamido)propanoic acid | 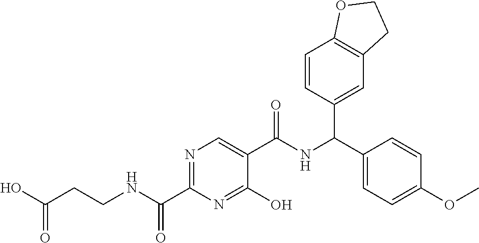 [SMILES: COc1ccc(C(NC(=O)c2cnc(C(=O)NCCC(=O)O)nc2O)c2ccc3c(c2)CCO3)cc1] | (M + 1)⁺ 493 IC50 1.7 nM |
| Ex. 40 | 3-(5-(((4-chlorophenyl)(4- (trifluoromethyl)phenyl) methyl)carbamoyl)-4- hydroxypyrimidine-2- carboxamido)propanoic acid | 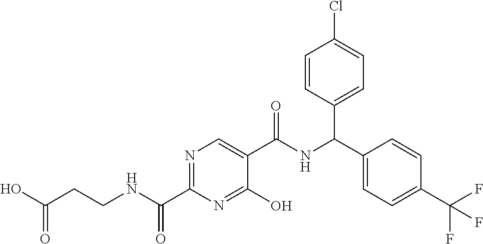 [SMILES: O=C(O)CCNC(=O)c1ncc(C(=O)NC(c2ccc(Cl)cc2)c2ccc(C(F)(F)F)cc2)c(O)n1] | (M + 1)⁺ 523 IC50 0.9 nM |
| Ex. 41 | 3-(5-((bis(6- methoxypyridin-3- yl)methyl)carbamoyl)-4- hydroxypyrimidine-2- carboxamido)propanoic acid | 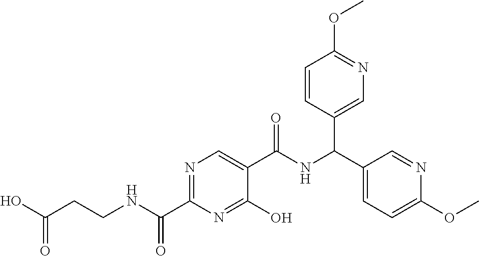 [SMILES: COc1ccc(C(NC(=O)c2cnc(C(=O)NCCC(=O)O)nc2O)c2ccc(OC)nc2)cn1] | (M + 1)⁺ 483 IC50 0.8 nM |
| Ex. 42 | 3-(5-((bis(4- chlorophenyl)methyl) carbamoyl)-4- hydroxypyrimidine-2- carboxamido)propanoic acid | 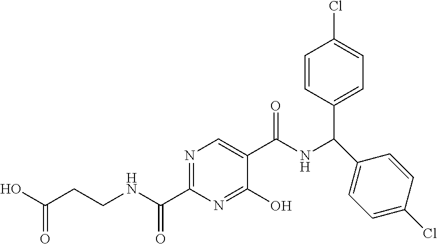 [SMILES: O=C(O)CCNC(=O)c1ncc(C(=O)NC(c2ccc(Cl)cc2)c2ccc(Cl)cc2)c(O)n1] | (M + 1)⁺ 489 IC50 0.5 nM |
| Ex. 43 | (R)-N5-(1-(4- bromophenyl)ethyl)-4- hydroxy-N2- propylpyrimidine-2,5- dicarboxamide | 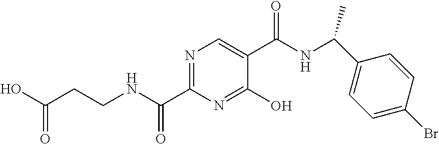 | (M + 1)⁺ 407 IC50 1.6 nM |
| Ex. 44 | 3-(5-((1-(4′-fluoro-[1,1′- biphenyl]-4- yl)ethyl)carbamoyl)-4- hydroxypyrimidine-2- carboxamido)propanoic acid | 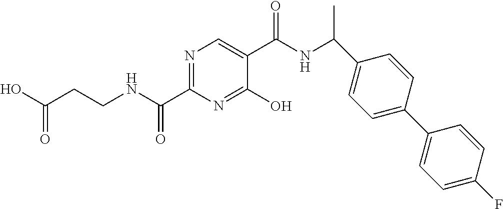 [SMILES: CC(NC(=O)c1cnc(C(=O)NCCC(=O)O)nc1O)c1ccc(-c2ccc(F)cc2)cc1] | (M + 1)⁺ 453 IC50 2.5 nM |
| Ex. 45 | 3-(5-((1-(4- bromophenyl)cyclopropyl) carbamoyl)-4- hydroxypyrimidine-2- carboxamido)propanoic acid | 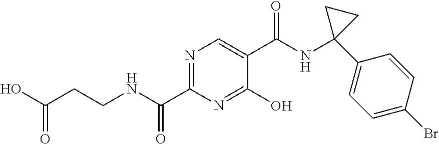 [SMILES: O=C(O)CCNC(=O)c1ncc(C(=O)NC2(c3ccc(Br)cc3)CC2)c(O)n1] | (M + 1)⁺ 451 IC50 0.4 nM |
| Ex. 46 | 3-(4-hydroxy-5-((1-(4- phenoxyphenyl)ethyl) carbamoyl)pyrimidine-2- carboxamido)propanoic acid | 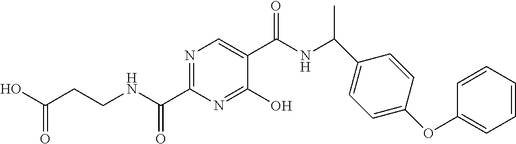 [SMILES: CC(NC(=O)c1cnc(C(=O)NCCC(=O)O)nc1O)c1ccc(Oc2ccccc2)cc1] | (M + 1)⁺ 451 IC50 1.0 nM |
| Ex. 47 | 3-(5-((1-(4-bromophenyl)- 2,2,2- trifluoroethyl)carbamoyl)-4- hydroxypyrimidine-2- carboxamido)propanoic acid | 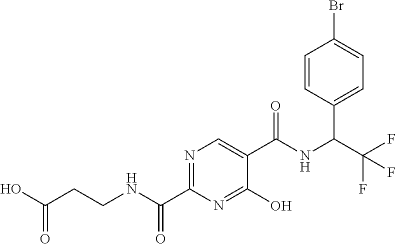 [SMILES: O=C(O)CCNC(=O)c1ncc(C(=O)NC(c2ccc(Br)cc2)C(F)(F)F)c(O)n1] | (M + 1)⁺ 491 IC50 1.1 nM |
| Ex. 48 | 3-(5-((bis(4- methoxyphenyl)methyl) carbamoyl)-4- hydroxypyrimidine-2- carboxamido)propanoic acid | 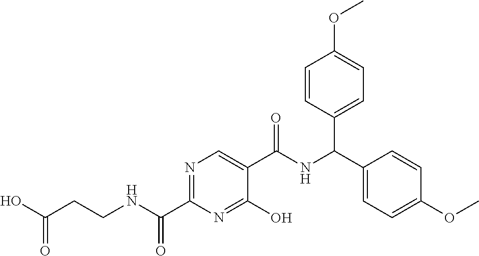 [SMILES: COc1ccc(C(NC(=O)c2cnc(C(=O)NCCC(=O)O)nc2O)c2ccc(OC)cc2)cc1] | (M + 1)⁺ 481 IC50 0.7 nM |
| Ex. 49 | 3-(4-hydroxy-5-((phenyl(4- (trifluoromethyl)phenyl) methyl)carbamoyl) pyrimidine-2- carboxamido)propanoic acid | 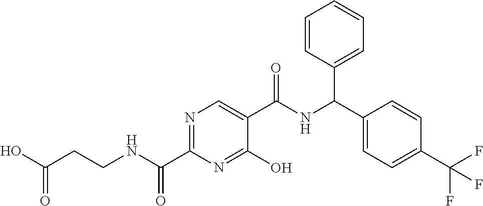 [SMILES: O=C(O)CCNC(=O)c1ncc(C(=O)NC(c2ccccc2)c2ccc(C(F)(F)F)cc2)c(O)n1] | (M + 1)⁺ 489 IC50 1.0 nM |

The half life of compounds described in this application were evaluated in rat. Shown in Table 9 are T½ of selected examples in rat (Wister Han) for selected compounds of the present invention. In addition Table 9 includes half life data for compounds disclosed in International Patent Application published as WO 2013/043621.

An increased plasma half-life is a desirable property, as it would be expected to lead to greater in vivo efficacy. The species of the present invention as seen in Table 9 demonstrate a favorable pharmacokinetic profile of longer half-lives in the rat, compared to structurally similar species exemplified previously (Examples 2-11, 2-12 and 2-16 in WO 2013/043621. As one can see from the data, substantial differences in rat half-lives were observed with subtle changes in chemical structure; these effects are unpredictable and unexpected.

The pharmacokinetics of the compounds disclosed in Table 9 were studied in male Wistar Han rats after intravenous (IV) administration. For IV dosing at 0.5 mg/kg in rat, compounds were formulated as a solution in DMSO/PEG400/water (20/60/20, by vol.). Plasma samples obtained from dosed animals were prepared for analysis by means of a single step protein precipitation technique by adding 200 μL of acetonitrile to 50 μL aliquots of individual subject samples. Samples were mixed by vortex for homogeneity and then subjected to centrifugation at 3500 rpm for 10 min. The supernatant (200 μL) was collected and injected into the LC-MS/MS for analysis. Pharmacokinetic parameters were calculated using established non-compartmental methods.

TABLE 9
Half Life of Selected Examples in Rat (Wistar Han)

| Example | | Half Life |
|---|---|---|
| Number | Chemical Structure | (t1/2) (Hr) |
| Example 2-11 from WO2013/040789 | 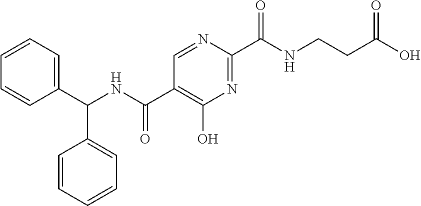 [SMILES: O=C(O)CCNC(=O)c1ncc(C(=O)NC(c2ccccc2)c2ccccc2)c(O)n1] | 0.28 |
| Example 2-12 from WO2013/040789 | 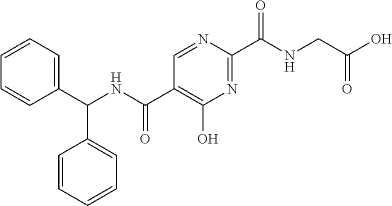 [SMILES: O=C(O)CNC(=O)c1ncc(C(=O)NC(c2ccccc2)c2ccccc2)c(O)n1] | 0.56 |
| Example 2-16 from WO2013/040789 | 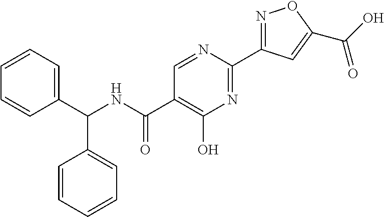 [SMILES: O=C(O)c1cc(-c2ncc(C(=O)NC(c3ccccc3)c3ccccc3)c(O)n2)no1] | 0.3 |
| Example 1 | 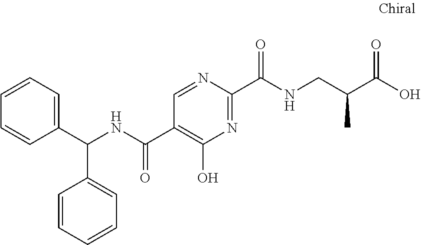 [SMILES: C[C@@H](CNC(=O)c1ncc(C(=O)NC(c2ccccc2)c2ccccc2)c(O)n1)C(=O)O] | 1.9 |
| Example 2 | 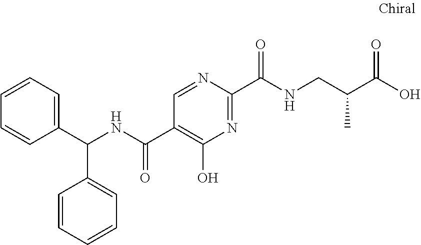 [SMILES: C[C@H](CNC(=O)c1ncc(C(=O)NC(c2ccccc2)c2ccccc2)c(O)n1)C(=O)O] | 1.45 |
| Example 4 | 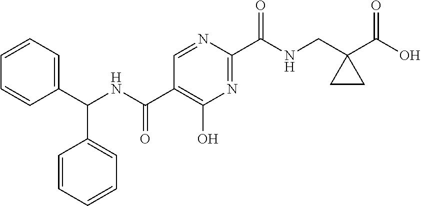 [SMILES: O=C(NCC1(C(=O)O)CC1)c1ncc(C(=O)NC(c2ccccc2)c2ccccc2)c(O)n1] | 4.78 |
| Example 6 | 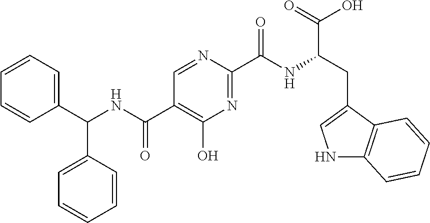 [SMILES: O=C(N[C@@H](Cc1c[nH]c2ccccc12)C(=O)O)c1ncc(C(=O)NC(c2ccccc2)c2ccccc2)c(O)n1] | 1.68 |
| Example 7 | 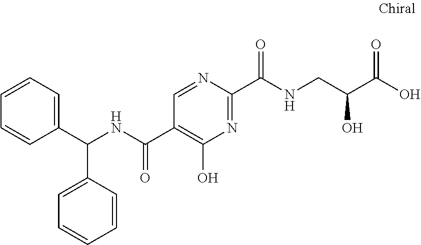 [SMILES: O=C(NC[C@H](O)C(=O)O)c1ncc(C(=O)NC(c2ccccc2)c2ccccc2)c(O)n1] | 1.02 |
| Example 14 | 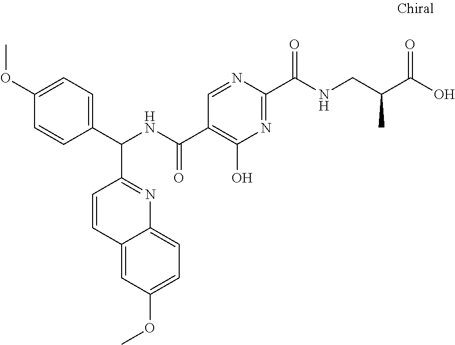 [SMILES: COc1ccc(C(NC(=O)c2cnc(C(=O)NC[C@H](C)C(=O)O)nc2O)c2ccc3cc(OC)ccc3n2)cc1] | 1.36 |
| Example 15 | 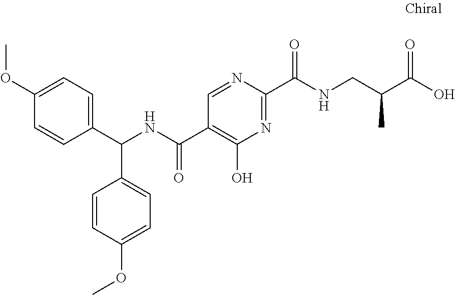 [SMILES: COc1ccc(C(NC(=O)c2cnc(C(=O)NC[C@H](C)C(=O)O)nc2O)c2ccc(OC)cc2)cc1] | 0.99 |
| Example 16 | 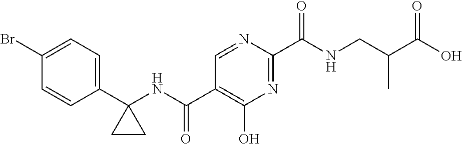 [SMILES: CC(CNC(=O)c1ncc(C(=O)NC2(c3ccc(Br)cc3)CC2)c(O)n1)C(=O)O] | 0.88 |
| Example 17 | 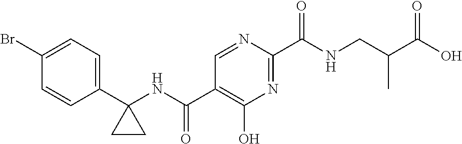 [SMILES: CC(CNC(=O)c1ncc(C(=O)NC2(c3ccc(Br)cc3)CC2)c(O)n1)C(=O)O] | 1.36 |
| Example 36 | 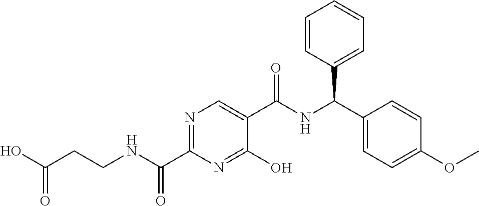 [SMILES: COc1ccc([C@@H](NC(=O)c2cnc(C(=O)NCCC(=O)O)nc2O)c2ccccc2)cc1] | 6.76 |
| Example 37 | 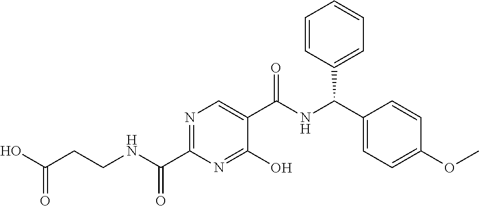 [SMILES: COc1ccc([C@H](NC(=O)c2cnc(C(=O)NCCC(=O)O)nc2O)c2ccccc2)cc1] | 0.69 |
| Example 39 | 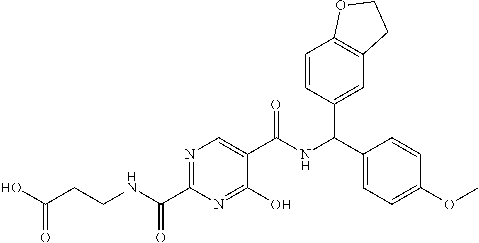 [SMILES: COc1ccc(C(NC(=O)c2cnc(C(=O)NCCC(=O)O)nc2O)c2ccc3c(c2)CCO3)cc1] | 1.59 |
| Example 43 | 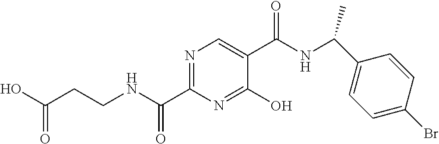 [SMILES: C[C@@H](NC(=O)c1cnc(C(=O)NCCC(=O)O)nc1O)c1ccc(Br)cc1] | 0.71 |
| Example 44 | 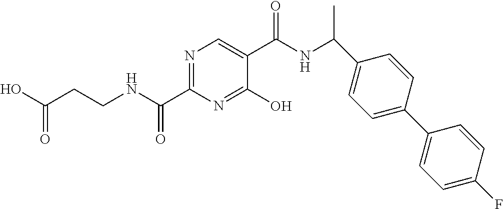 [SMILES: CC(NC(=O)c1cnc(C(=O)NCCC(=O)O)nc1O)c1ccc(-c2ccc(F)cc2)cc1] | 0.69 (racemate) |
| Example 45 | 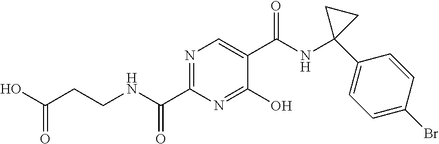 [SMILES: O=C(O)CCNC(=O)c1ncc(C(=O)NC2(c3ccc(Br)cc3)CC2)c(O)n1] | 0.62 |

1. (canceled)

2. (canceled)

3. (canceled)

4. (canceled)

5. (canceled)

6. (canceled)

7. (canceled)

8. (canceled)

9. A compound according to claim 1, or a stereoisomer thereof, or a pharmaceutically acceptable salt thereof, selected from:
(R)-3-(5-(Benzhydrylcarbamoyl)-4-hydroxypyrimidine-2-carboxamido)-2-methyl propanoic acid;
(S)-3-(5-(Benzhydrylcarbamoyl)-4-hydroxypyrimidine-2-carboxamido)-2-methyl propanoic acid;
3-(5-(Benzhydrylcarbamoyl)-4-hydroxypyrimidine-2-carboxamido)-2-hydroxy-2-methylpropanoic acid;
1-((5-(benzhydrylcarbamoyl)-4-hydroxypyrimidine-2-carboxamido)methyl)cyclopropanecarboxylic acid;
3-(5-(benzhydrylcarbamoyl)-4-hydroxypyrimidine-2-carboxamido)-2-hydroxypropanoic acid;
(2S)-2-(5-(benzhydrylcarbamoyl)-4-hydroxypyrimidine-2-carboxamido)-3-(3aH-indol-3-yl)propanoic acid;
(2R)-2-(5-(benzhydrylcarbamoyl)-4-hydroxypyrimidine-2-carboxamido)-3-(3aH-indol-3-yl)propanoic acid;
2-(5-(benzhydrylcarbamoyl)-4-hydroxypyrimidine-2-carboxamido)-3-(3aH-indol-3-yl)propanoic acid;
(S)-3-(5-(benzhydrylcarbamoyl)-4-hydroxypyrimidine-2-carboxamido)-2-hydroxypropanoic acid;
(R)-3-(5-(benzhydrylcarbamoyl)-4-hydroxypyrimidine-2-carboxamido)-2-hydroxypropanoic acid;
3-(5-(benzhydrylcarbamoyl)-4-hydroxypyrimidine-2-carboxamido)-2-hydroxypropanoic acid;
(R)-3-(5-(((4-Cyanophenyl)(phenyl)methyl)carbamoyl)-4-hydroxypyrimidine-2-carboxamido)-2-hydroxypropanoic acid;
(S)-3-(5-(((4-Cyanophenyl)(phenyl)methyl)carbamoyl)-4-hydroxypyrimidine-2-carboxamido)-2-hydroxypropanoic acid;
3-(5-(((4-Cyanophenyl)(phenyl)methyl)carbamoyl)-4-hydroxypyrimidine-2-carboxamido)-2-hydroxypropanoic acid;
(R)-2-hydroxy-3-(4-hydroxy-5-(((S)-(4-methoxyphenyl) (phenyl)methyl)carbamoyl)pyrimidine-2-carboxamido)propanoic acid;
(R)-2-hydroxy-3-(4-hydroxy-5-((2-(4-(trifluoromethyl)phenyl)propan-2-yl)carbamoyl)pyrimidine-2-carboxamido)propanoic acid;
(S)-2-hydroxy-3-(4-hydroxy-5-((2-(4-(trifluoromethyl)phenyl)propan-2-yl)carbamoyl)pyrimidine-2-carboxamido)propanoic acid;
2-hydroxy-3-(4-hydroxy-5-((2-(4-(trifluoromethyl)phenyl)propan-2-yl)carbamoyl)pyrimidine-2-carboxamido)propanoic acid;
(R)-2-hydroxy-3-(4-hydroxy-5-(((R)-(4-methoxyphenyl) (phenyl)methyl)carbamoyl)pyrimidine-2-carboxamido)propanoic acid;
2-hydroxy-3-(4-hydroxy-5-(((4-methoxyphenyl) (phenyl)methyl)carbamoyl)pyrimidine-2-carboxamido)propanoic acid;
3-(5-((Bis(4-chlorophenyl)methyl)carbamoyl)-4-hydroxypyrimidine-2-carboxamido)-2-methylpropanoic acid;
3-(4-hydroxy-5-(((4-methoxyphenyl)(6-methoxyquinolin-2-yl)methyl)carbamoyl)pyrimidine-2-carboxamido)-2-methylpropanoic acid;
(R)-3-(4-hydroxy-5-(((4-methoxyphenyl)(6-methoxyquinolin-2-yl)methyl)carbamoyl)pyrimidine-2-carboxamido)-2-methylpropanoic acid;
(S)-3-(4-hydroxy-5-(((4-methoxyphenyl)(6-methoxyquinolin-2-yl)methyl)carbamoyl)pyrimidine-2-carboxamido)-2-methylpropanoic acid;
3-(5-((bis(4-methoxyphenyl)methyl)carbamoyl)-4-hydroxypyrimidine-2-carboxamido)-2-methylpropanoic acid;
(R)-3-(5-((bis(4-methoxyphenyl)methyl)carbamoyl)-4-hydroxypyrimidine-2-carboxamido)-2-methylpropanoic acid;
(S)-3-(5-((bis(4-methoxyphenyl)methyl)carbamoyl)-4-hydroxypyrimidine-2-carboxamido)-2-methylpropanoic acid;
3-(5-((1-(4-bromophenyl)cyclopropyl)carbamoyl)-4-hydroxypyrimidine-2-carboxamido)-2-methylpropanoic acid;
(S)-3-(5-((1-(4-bromophenyl)cyclopropyl)carbamoyl)-4-hydroxypyrimidine-2-carboxamido)-2-methylpropanoic acid;
(R)-3-(5-((1-(4-bromophenyl)cyclopropyl)carbamoyl)-4-hydroxypyrimidine-2-carboxamido)-2-methylpropanoic acid;
3-(5-((bis(4-methoxyphenyl)methyl)carbamoyl)-4-hydroxypyrimidine-2-carboxamido)-2-methylpropanoic acid;
(S)-3-(5-((bis(4-methoxyphenyl)methyl)carbamoyl)-4-hydroxypyrimidine-2-carboxamido)-2-methylpropanoic acid;
(R)-3-(5-((bis(4-methoxyphenyl)methyl)carbamoyl)-4-hydroxypyrimidine-2-carboxamido)-2-methylpropanoic acid;
3-(4-hydroxy-5-((2-(6-(4-methoxyphenyl)pyridin-3-yl)propan-2-yl)carbamoyl)pyrimidine-2-carboxamido)-2-methylpropanoic acid;
(R)-3-(4-hydroxy-5-((2-(6-(4-methoxyphenyl)pyridin-3-yl)propan-2-yl)carbamoyl)pyrimidine-2-carboxamido)-2-methylpropanoic acid;
(S)-3-(4-hydroxy-5-((2-(6-(4-methoxyphenyl)pyridin-3-yl)propan-2-yl)carbamoyl)pyrimidine-2-carboxamido)-2-methylpropanoic acid;
3-(4-hydroxy-5-((2-(4′-(trifluoromethyl)-[1,1′-biphenyl]-4-yl)propan-2-yl)carbamoyl)pyrimidine-2-carboxamido)-2-methylpropanoic acid;
2-((2-carboxypropyl)carbamoyl)-6-hydroxy-5-((2-(4-(isoquinolin-5-yl)phenyl)propan-2-yl)carbamoyl)pyrimidin-1-ium formate;
(S)-2-((2-carboxypropyl)carbamoyl)-6-hydroxy-5-((2-(4-(isoquinolin-5-yl)phenyl)propan-2-yl)carbamoyl)pyrimidin-1-ium formate;
(R)-2-((2-carboxypropyl)carbamoyl)-6-hydroxy-5-((2-(4-(isoquinolin-5-yl)phenyl)propan-2-yl)carbamoyl)pyrimidin-1-ium formate;
2-((2-carboxypropyl)carbamoyl)-6-hydroxy-5-((2-(4-(pyridin-3-yl)phenyl)propan-2-yl)carbamoyl)pyrimidin-1-ium formate;
(R)-2-((2-carboxypropyl)carbamoyl)-6-hydroxy-5-((2-(4-(pyridin-3-yl)phenyl)propan-2-yl)carbamoyl)pyrimidin-1-ium formate;
(S)-2-((2-carboxypropyl)carbamoyl)-6-hydroxy-5-((2-(4-(pyridin-3-yl)phenyl)propan-2-yl)carbamoyl)pyrimidin-1-ium formate;
3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
(R)-3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
(S)-3-(5-((2-(4′-cyano-2′-methyl-[1,1′-biphenyl]-4-yl)propan-2-yl)carbamoyl)-4-hydroxypyrimidine-2-carboxamido)-2-methylpropanoic acid;
3-(5-((2-(4′-fluoro-[1,1′-biphenyl]-4-yl)propan-2-yl)carbamoyl)-4-hydroxypyrimidine-2-carboxamido)-2-methylpropanoic acid;
(R)-3-(5-((2-(4′-fluoro-[1,1′-biphenyl]-4-yl)propan-2-yl)carbamoyl)-4-hydroxypyrimidine-2-carboxamido)-2-methylpropanoic acid;
(S)-3-(5-((2-(4′-fluoro-[1,1′-biphenyl]-4-yl)propan-2-yl)carbamoyl)-4-hydroxypyrimidine-2-carboxamido)-2-methylpropanoic acid;
3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(R)-3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(S)-3-(4-hydroxy-5-((2-(4′-(trifluoromethoxy)-[1,1′-biphenyl]-4-yl)propan-2-yl)carbamoyl)pyrimidine-2-carboxamido)-2-methylpropanoic acid;
(R)-2-((2-carboxypropyl)carbamoyl)-6-hydroxy-5-((2-(4-(6-methoxypyridin-3-yl)phenyl)propan-2-yl)carbamoyl)pyrimidin-1-ium chloride;
2-((2-carboxypropyl)carbamoyl)-6-hydroxy-5-((2-(4-(6-methoxypyridin-3-yl)phenyl)propan-2-yl)carbamoyl)pyrimidin-1-ium chloride;
(S)-2-((2-carboxypropyl)carbamoyl)-6-hydroxy-5-((2-(4-(6-methoxypyridin-3-yl)phenyl)propan-2-yl)carbamoyl)pyrimidin-1-ium chloride;
5-((2-(4-(1H-pyrazol-3-yl)phenyl)propan-2-yl)carbamoyl)-2-((2-carboxypropyl)carbamoyl)-6-hydroxypyrimidin-1-ium formate;
(R)-5-((2-(4-(1H-pyrazol-3-yl)phenyl)propan-2-yl)carbamoyl)-2-((2-carboxypropyl)carbamoyl)-6-hydroxypyrimidin-1-ium formate;
(S)-5-((2-(4-(1H-pyrazol-3-yl)phenyl)propan-2-yl)carbamoyl)-2-((2-carboxypropyl)carbamoyl)-6-hydroxypyrimidin-1-ium formate;
3-(5-((2-(4′-chloro-[1,1′-biphenyl]-4-yl)propan-2-yl)carbamoyl)-4-hydroxypyrimidine-2-carboxamido)-2-methylpropanoic acid;
(R)-3-(5-((2-(4′-chloro-[1,1′-biphenyl]-4-yl)propan-2-yl)carbamoyl)-4-hydroxypyrimidine-2-carboxamido)-2-methylpropanoic acid;
(S)-3-(5-((2-(4′-chloro-[1,1′-biphenyl]-4-yl)propan-2-yl)carbamoyl)-4-hydroxypyrimidine-2-carboxamido)-2-methylpropanoic acid;
3-(4-hydroxy-5-((1-(4′-(trifluoromethyl)-[1,1′-biphenyl]-4-yl)cyclopropyl) carbamoyl)pyrimidine-2-carboxamido)-2-methyl propanoic acid;
(R)-3-(4-hydroxy-5-((1-(4′-(trifluoromethyl)-[1,1′-biphenyl]-4-yl)cyclopropyl) carbamoyl)pyrimidine-2-carboxamido)-2-methyl propanoic acid;
(S)-3-(4-hydroxy-5-((1-(4′-(trifluoromethyl)-[1,1′-biphenyl]-4-yl)cyclopropyl) carbamoyl)pyrimidine-2-carboxamido)-2-methyl propanoic acid;
3-(5-((1-(4′-chloro-[1,1′-biphenyl]-4-yl)cyclopropyl)carbamoyl)-4-hydroxypyrimidine-2-carboxamido)-2-methylpropanoic acid;
(R)-3-(5-((1-(4′-chloro-[1,1′-biphenyl]-4-yl)cyclopropyl)carbamoyl)-4-hydroxypyrimidine-2-carboxamido)-2-methylpropanoic acid;
(S)-3-(5-((1-(4′-chloro-[1,1′-biphenyl]-4-yl)cyclopropyl)carbamoyl)-4-hydroxypyrimidine-2-carboxamido)-2-methylpropanoic acid;
2-((2-carboxypropyl)carbamoyl)-6-hydroxy-5-((1-(4-(isoquinolin-5-yl)phenyl)cyclopropyl)carbamoyl)pyrimidin-1-ium formate;
(S)-2-((2-carboxypropyl)carbamoyl)-6-hydroxy-5-((1-(4-(isoquinolin-5-yl)phenyl)cyclopropyl)carbamoyl)pyrimidin-1-ium formate;
(R)-2-((2-carboxypropyl)carbamoyl)-6-hydroxy-5-((1-(4-(isoquinolin-5-yl)phenyl)cyclopropyl)carbamoyl)pyrimidin-1-ium formate;
3-(5-((1-(4′-fluoro-[1,1′-biphenyl]-4-yl)cyclopropyl)carbamoyl)-4-hydroxypyrimidine-2-carboxamido)-2-methylpropanoic acid;
(R)-3-(5-((1-(4′-fluoro-[1,1′-biphenyl]-4-yl)cyclopropyl)carbamoyl)-4-hydroxypyrimidine-2-carboxamido)-2-methylpropanoic acid;
(S)-3-(5-((1-(4′-fluoro-[1,1′-biphenyl]-4-yl)cyclopropyl)carbamoyl)-4-hydroxypyrimidine-2-carboxamido)-2-methylpropanoic acid;
2-((2-carboxypropyl)carbamoyl)-6-hydroxy-5-((1-(4-(6-methoxypyridin-3-yl)phenyl)cyclopropyl)carbamoyl)pyrimidin-1-ium chloride;
(R)-2-((2-carboxypropyl)carbamoyl)-6-hydroxy-5-((1-(4-(6-methoxypyridin-3-yl)phenyl)cyclopropyl)carbamoyl)pyrimidin-1-ium chloride;
(S)-2-((2-carboxypropyl)carbamoyl)-6-hydroxy-5-((1-(4-(6-methoxypyridin-3-yl)phenyl)cyclopropyl)carbamoyl)pyrimidin-1-ium chloride;
3-(4-hydroxy-5-(((4-methoxyphenyl)(6-(methylamino)pyridin-3-yl)methyl)carbamoyl)pyrimidine-2-carboxamido)propanoic acid;
(R)-3-(4-hydroxy-5-(((4-methoxyphenyl)(6-(methylamino)pyridin-3-yl)methyl)carbamoyl)pyrimidine-2-carboxamido)propanoic acid;
(S)-3-(4-hydroxy-5-(((4-methoxyphenyl)(6-(methylamino)pyridin-3-yl)methyl)carbamoyl)pyrimidine-2-carboxamido)propanoic acid;
3-(4-hydroxy-5-(((4-methoxyphenyl)(phenyl)methyl)carbamoyl)pyrimidine-2-carboxamido)propanoic acid;
(S)-3-(4-hydroxy-5-(((4-methoxyphenyl)(phenyl)methyl)carbamoyl)pyrimidine-2-carboxamido)propanoic acid;
(S)-3-(5-(((4-chlorophenyl)(4-methoxyphenyl)methyl)carbamoyl)-4-hydroxypyrimidine-2-carboxamido)propanoic acid;
(R)-3-(5-(((4-chlorophenyl)(4-methoxyphenyl)methyl)carbamoyl)-4-hydroxypyrimidine-2-carboxamido)propanoic acid;
3-(5-(((4-chlorophenyl)(4-methoxyphenyl)methyl)carbamoyl)-4-hydroxypyrimidine-2-carboxamido)propanoic acid;
3-(5-(((2,3-dihydrobenzofuran-5-yl)(4-methoxyphenyl)methyl)carbamoyl)-4-hydroxypyrimidine-2-carboxamido)propanoic acid;
3-(5-(((4-chlorophenyl)(4-(trifluoromethyl)phenyl)methyl)carbamoyl)-4-hydroxypyrimidine-2-carboxamido)propanoic acid
3-(5-((bis(6-methoxypyridin-3-yl)methyl)carbamoyl)-4-hydroxypyrimidine-2-carboxamido)propanoic acid;
3-(5-((bis(4-chlorophenyl)methyl)carbamoyl)-4-hydroxypyrimidine-2-carboxamido)propanoic acid;
(R)-N5-(1-(4-bromophenyl)ethyl)-4-hydroxy-N2-propylpyrimidine-2,5-dicarboxamide;
N5-(1-(4-bromophenyl)ethyl)-4-hydroxy-N2-propylpyrimidine-2,5-dicarboxamide;
3-(5-((1-(4′-fluoro-[1,1′-biphenyl]-4-yl)ethyl)carbamoyl)-4-hydroxypyrimidine-2-carboxamido)propanoic acid;
3-(5-((1-(4-bromophenyl)cyclopropyl)carbamoyl)-4-hydroxypyrimidine-2-carboxamido)propanoic acid;
3-(4-hydroxy-5-((1-(4-phenoxyphenyl)ethyl)carbamoyl)pyrimidine-2-carboxamido)propanoic acid;
3-(5-((1-(4-bromophenyl)-2,2,2-trifluoroethyl)carbamoyl)-4-hydroxypyrimidine-2-carboxamido) propanoic acid;
3-(5-((bis(4-methoxyphenyl)methyl)carbamoyl)-4-hydroxypyrimidine-2-carboxamido)propanoic acid; and
3-(4-hydroxy-5-((phenyl(4-(trifluoromethyl)phenyl)methyl)carbamoyl)pyrimidine-2-carboxamido) propanoic acid.

10. A compound of claim 9 or a pharmaceutically acceptable salt thereof, for use as a medicament.

11. A compound of claim 9 or a pharmaceutically acceptable salt thereof, for the treatment of conditions mediated by HIF prolyl hydroxylase.

12. A pharmaceutical composition comprising a compound of claim 9 and pharmaceutically acceptable carrier.

13. A method of enhancing endogenous production of erythropoietin in a mammal which comprises administering to the mammal an amount of a compound of claim 9, or a pharmaceutically acceptable salt or solvate thereof, that is effective for enhancing endogenous production of erythropoietin.

14. A method for the treatment of anemia in a mammal which comprises administering to the mammal an effective amount of a compound of claim 9, or a pharmaceutically acceptable salt or solvate thereof.

15. A method for the prevention of anemia in a mammal which comprises administering to the mammal an effective amount of a compound of claim 9, or a pharmaceutically acceptable salt or solvate thereof.

16. Use of a compound of claim 9, or a pharmaceutically acceptable salt or solvate thereof, in the manufacture of medicaments for the treatment of conditions mediated by HIF prolyl hydroxylase.
